SEQUENCE LISTING (29 sequences)

SEQ ID NO: 1 - protein, 20 aa, Homo sapiens
  DOMAIN (1)..(20): Domain of p53 (304)...(323)
TKRALPNNTSSSPQPKKKPL

SEQ ID NO: 2 - protein, 25 aa, Virus; Host Homo sapiens
  DOMAIN (1)..(25): Domain of papillomavirus E1 protein (84)...(108)
KRKVLGSSQNSSGSEASETPVKRRK

SEQ ID NO: 3 - protein, 13 aa, Virus; Host Macaca mulatta
  DOMAIN (1)..(13): Domain of SV40 T-Antigen Protein (118)...(130)
ADSQHSTPPKKKR

SEQ ID NO: 4 - protein, 13 aa, Virus; Host Homo sapiens
  DOMAIN (1)..(13): Domain HPV11 Initiator Protein (462)...(474)
KLWLHGTPKKNCI

SEQ ID NO: 5 - protein, 13 aa, Virus; Host Homo sapiens
  DOMAIN (1)..(13): Domain of HPV18 Initiator Protein (468)...(480)
KSFLKGTPKKNCL

SEQ ID NO: 6 - protein, 12 aa, Virus; Host Homo sapiens
  DOMAIN (1)..(12): Domain of JCV T-Antigen (120)...(131)
GSQHSTPPKKKR

SEQ ID NO: 7 - protein, 13 aa, Virus; Host Cercopithecus aethiops
  DOMAIN (1)..(13): Domain of LPV Initiator Protein (181)...(193)
QSSYTCTPPKRKK

SEQ ID NO: 8 - protein, 13 aa, Virus; Host Mesocricetus auratus
  DOMAIN (1)..(13): Domain of HaPV Initiator Protein (234)...(255)
QQSHHNTTPKKPP

SEQ ID NO: 9 - protein, 13 aa, Virus; Host Mus musculus
  DOMAIN (1)..(13): Domain of PyV Initiator Protein (272)...(284)
QSSFNATPPKKAR

SEQ ID NO: 10 - protein, 15 aa, Virus; Host Mus musculus
  DOMAIN (1)..(15): Domain of KV Initiator Protein (134)...(146)
PQRSSSQATPPKKKA

SEQ ID NO: 11 - protein, 17 aa, Virus; Host Macaca mulatta
  DOMAIN (1)..(17): Domain SV40 T-Antigen (116)...(132)
ATADSQHSTPPKKKRKV

SEQ ID NO: 12 - protein, 13 aa, Artificial Sequence
  Description of Artificial Sequence Synthetic Peptide
ADSQHSTPPKKKR

SEQ ID NO: 13 - protein, 13 aa, Artificial Sequence
  Description of Artificial Sequence Synthetic Peptide
PPKKKRTADSQHS

SEQ ID NO: 14 - protein, 13 aa, Artificial Sequence
  Description of Artificial Sequence Synthetic Peptide
PPKKKRTADSQHS

SEQ ID NO: 15 - protein, 17 aa, Artificial Sequence
  Description of Artificial Sequence Synthetic Peptide
ATADSQHSTPPKKKRKV

SEQ ID NO: 16 - protein, 17 aa, Artificial Sequence
  Description of Artificial Sequence Synthetic Peptide
PPKKKRKVTATADSQHS

SEQ ID NO: 17 - protein, 17 aa, Artificial Sequence
  Description of Artificial Sequence Synthetic Peptide
PPKKKRKVTATADSQHS

SEQ ID NO: 18 - protein, 25 aa, Artificial Sequence
  Description of Artificial Sequence Synthetic Peptide
KRKVLGSSQNSSGSEASETPVKRRK

SEQ ID NO: 19 - protein, 25 aa, Artificial Sequence
  Description of Artificial Sequence Synthetic Peptide
SEASETPVKRRKGKRKVLGSSQNSS

SEQ ID NO: 20 - protein, 13 aa, Artificial Sequence
  Description of Artificial Sequence Synthetic Peptide
ADSQHSAPPKKKR

SEQ ID NO: 21 - protein, 13 aa, Artificial Sequence
  Description of Artificial Sequence Synthetic Peptide
ADSQHSTAPKKKR

SEQ ID NO: 22 - protein, 13 aa, Artificial Sequence
  Description of Artificial Sequence Synthetic Peptide
ADSQHSTPAKKKR

SEQ ID NO: 23 - protein, 13 aa, Artificial Sequence
  Description of Artificial Sequence Synthetic Peptide
ADSQHSTAAKKKR

SEQ ID NO: 24 - DNA, 64 bp, Artificial Sequence
  Description of Artificial Sequence Synthetic Nucleic Acid
taattttttttatttatgcagaggccgaggccgcctcggcctctgagctattccagaagtagtg

SEQ ID NO: 25 - protein, 9 aa, Artificial Sequence
  Description of Artificial Sequence Synthetic Peptide
SQHSTPPKK

SEQ ID NO: 26 - protein, 9 aa, Artificial Sequence
  Description of Artificial Sequence Synthetic Peptide
SQHSTPPKK

SEQ ID NO: 27 - protein, 9 aa, Artificial Sequence
  Description of Artificial Sequence Synthetic Peptide
PPKKTSQHS

SEQ ID NO: 28 - protein, 9 aa, Artificial Sequence
  Description of Artificial Sequence Synthetic Peptide
PPKKTSQHS

SEQ ID NO: 29 - DNA, 64 bp, Artificial Sequence
  Description of Artificial Sequence Synthetic Nucleic Acid
gagatgcatgctttgcatacttctgcctgctggggagggaattcttgaagacgaaagggcctcg

(19) United States
(12) Patent Application Publication
(10) Pub. No.: US 2004/0147006 A1
(43) Pub. Date: Jul. 29, 2004

(54) Peptides that bind to DNA and inhibit DNA replication, and methods of use

(76) Inventor: Peter Bullock, Boston, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/701,778

(22) Filed: Nov. 5, 2003

Related U.S. Application Data
(63) Continuation-in-part of application No. PCT/US02/14372, filed on May 7, 2002.
(60) Provisional application No. 60/289,084, filed on May 7, 2001.
(60) Provisional application No. 60/344,706, filed on Oct. 24, 2001.

(51) Int. Cl. C12N 9/22; C07H 21/04

(52) U.S. Cl. 435/199; 435/69.1; 435/320.1; 435/325; 536/23.2

(57) ABSTRACT

Isolated peptides are provided that span the Simian Virus 40 DNA region of the T-ag (or the Bovine Papillomavirus E1 protein) centered on position Thr124 (or Thr102, respectively), which include regulatory motifs, such as the nuclear localization signal (NLS) and the consensus recognition site for cyclin-Cdk kinases. When unphosphorylated, peptides derived from this region bind to DNA and inhibit T-ag assembly. Upon phosphorylation at Thr124 (or Thr102), these peptides neither bind to DNA nor to inhibit T-ag assembly. These forms of regulation of DNA replication and T-ag assembly provide a novel target, and provide screens for anti-viral agents, and for gene therapy.

### RELATED APPLICATIONS

This application claims the benefit of PCT application US02/14372 filed May 7, 2002, and provisional applications 60/289,084 filed May 7, 2001 and 60/344,706 filed Oct. 24, 2001, all of which are hereby incorporated by reference herein in their entireties.

### GOVERNMENT FUNDING

This invention was made in part with government support under grant 9R01GM55397 awarded by the National Institutes of Health. The government has certain rights in the invention.

### FIELD OF THE INVENTION

The invention relates to peptides and peptide derivatives that bind to a DNA target and inhibit DNA replication of a virus, or DNA replication of a cell, having the DNA target.

### BACKGROUND

Initiation of genomic DNA replication is not well understood due to a paucity of information concerning DNA sequences of eukaryotic origins of replication (Burhans, W., et al., 1994, Science 263:639-640). In contrast to cellular genomic origins of DNA replication, the SV40 origin has been extensively characterized. Initiation of SV40 DNA replication requires the viral initiator protein termed T-antigen (T-ag), and other proteins supplied by the host (Tooze, J., 1981, DNA Tumor Viruses., 2nd ed. Cold Spring Harbor Laboratory, Cold Spring Harbor, N.Y.). T-ag is composed of 708 amino acids, has a molecular weight of 82 kDa, and plays numerous roles during initiation and elongation of viral DNA replication (Bullock, P. A., 1997, Critical Rev. Biochem. Molec. Biol. 32:503-568).

### SUMMARY OF THE INVENTION

The invention in various embodiments provides peptide compounds that bind to nucleic acids, for example to DNA, and inhibit replication. Binding of the peptide can be reversible and under specific control. The peptides are useful as regulatory elements in gene therapy, and for other in vivo regulatory functions.

An aspect of the invention features an isolated peptide comprising an amino acid sequence having a nuclear localization signal (NLS), which has a length of at least about 8 amino acids to about 50 amino acids, and which binds directly to a DNA molecule.

The peptide inhibits replication of the DNA molecule, for example, by binding to a DNA that contains an origin of replication. The peptide can inhibit transcription of the DNA molecule. The target DNA molecule can include a DNA sequence of either a eukaryotic or a viral gene. For example, the target DNA molecule has a DNA sequence from a papovavirus, such as a papillomavirus, for example a DNA sequence of a papillomavirus strain 16 or 18, which are among the more virulent papilloma strains. Further, the target DNA molecule can contain a sequence from a different pathogenic virus, for example, a DNA sequence from a BK virus or a JC virus. Alternatively, the target DNA molecule can contain a eukaryotic gene DNA sequence.

The peptide can include a cyclin/Cdk recognition site, and the peptide in these embodiments is not generally phosphorylated. Further, the amino acid sequence of some embodiments does not have a threonine, a serine, or a tyrosine, amino acid residues that are capable of being phosphorylated. Alternatively, the peptide includes a cyclin/Cdk recognition site, for example, a cyclin/Cdk recognition site having a serine (S) or a threonine (T) residue, and the residue is or is not phosphorylated.

The NLS sequence can be a naturally occurring sequence from, for example, a viral source, from a yeast, or from a vertebrate source, for example, a mammalian source. The NLS can be obtained from a variety of virus genes, for example, from the papillomavirus E1 gene, from SV40 T-Ag or VP2 genes, or other virus such as BKV, JCV, PLV, HaPV, PyV, or KV. Alternatively, the peptide includes an NLS sequence from a eukaryotic gene, such as p53, c-myc, c-myb, N-myc, lamin A, PDGF A chain, or Hsp 70. Generally, the peptide is produced by chemical synthesis. The NLS can be designed and synthesized, and can be mutagenized i.e., variants of the amino acid sequence are designed and optimized to obtain peptides having improved affinity for a DNA target by methods disclosed herein. In other embodiments, the peptide has one or more chemical modifications, for example, a modication which is an amino acid analog such as a D-amino acid substituted for an L-amino acid, or a modification is a non-peptidic bond. The peptide is formulated with a pharmaceutically acceptable carrier, for example, for administration to a mammalian subject such as a human.

In another aspect, the invention provides an isolated peptide having an amino acid sequence selected from the group consisting of:

| | |
|---|---|
| TKRALPNNTSSSPQPKKKPL, | (SEQ ID NO: 1) |
| KRKVLGSSQNSSGSEASETPVKRRK, | (SEQ ID NO: 2) |
| KKRRKRVKLVGPSTSEQSNASESSG, | (SEQ ID NO: 30) |
| ADSQHSTPPKKKR, | (SEQ ID NO: 3) |
| KLWLHGTPKKNCI, | (SEQ ID NO: 4) |
| KSFLKGTPKKNCL, | (SEQ ID NO: 5) |
| GSQHSTPPKKKR, | (SEQ ID NO: 6) |
| QSSYTCTPPKRKK, | (SEQ ID NO: 7) |
| QQSHHNTTPKKPP, | (SEQ ID NO: 8) |
| QSSFNATPPKKAR, | (SEQ ID NO: 9) |
| PARSQATPPKKKA, | (SEQ ID NO: 10) |
| ATADSQHSTPPKKKRKV; | (SEQ ID NO: 11) |
| SQHSTPPKK; | (SEQ ID NO: 26) |
| PPKKTSQHS; | (SEQ ID NO: 28) |
| ADSQHSDPPKKKR; | (SEQ ID NO: 73) |
| and | |
| ADSQHSTPPKAKR. | (SEQ ID NO: 74) |

In another aspect, the invention provides the NLS containing peptides comprising the amino acid sequences: KRKVLGSSQNSSGSEASETPVKRRK (SEQ ID NO: 2); ADSQHSTPPKKKR (SEQ ID NO: 3); an isolated peptide-comprising the amino acid sequence ATADSQHSTPPKKKRKV (SEQ ID NO: 11); and an isolated peptide comprising the amino acid sequence KKRRKRVKLVGPSTSEQSNASESSG (SEQ ID NO: 30), which is a rearranged version of SEQ ID NO: 2. Also provided is a polyvalent version of the peptide (a multimer), which has direct tandem repeats of all or a part of an amino acid sequence of the peptide such that the amino acid sequence is a “dimer” or a “trimer”. The peptides provided herein can be used in methods herein to inhibit DNA replication of a target DNA virus, by contacting a nucleic acid sequence of a naturally occurring viral DNA molecule with the provided peptide.

As the peptides provided herein optionally contain a site for cyclin/Cdk phosphorylation, in the unphosphorylated condition the peptide can bind directly to and inhibit replication or transcription of the DNA molecule. Further, the peptide can form a complex with the DNA molecule and inhibits assembly of a multimeric DNA-binding protein. The peptide with the cyclin/Cdk site can have a consensus sequence-T/S-P₁₋₂-K/R₂₋₄, wherein T/S is threonine or serine, P₁₋₂ is one to two amino acids at least one of which is a proline, and K/R₂₋₄ is two to four amino acids, at least two of which are lysine or arginine residues. An example of this consensus sequence is X₁-X₂-T/S-P/X₁₋₂-K/R₂₋₄ wherein X₁ and X₂ are two amino acid residues at the amino terminus, and P/X₁₋₂ is two amino acids at least one of which is a proline. Additional examples can be described as: T-P/X-K/R-K/R; T-P/X-P/X-K/R-K/R; T-P/X-K/R/X-K/R/X-K/R/X; T-P/X-P/X-K/R/X-K/R/X-K/R/X; T-P/X-K/R/X-K/R/X-K/R/X-K/R/X; and T-P/X-P/X-K/R/X-K/R/X-K/R/X-K/R/X, wherein K/R/X is an amino acid residue which can be a lysine, an arginine, or another amino acid. The synthetic peptides herein having an NLS sequence and lacking a functional Cdk site, i.e., lacking the residues T or an S, bind to the DNA molecule and are functionally equivalent.

These NLS consensus sequences described supra are “monopartite”, which means that the peptide has a single region having a sequence of two to four basic amino acid residues in the amino acid sequence. Alternatively, peptides provided herein have NLS sequences that are “bipartite”, i.e., the peptide has two regions of highly basic amino acid residues separated by a plurality of substantially non-basic amino acid residues. Such an NLS sequence is embodied by a consensus sequence K/R₂₋₄-Xₙ-T/S-P/X₁₋₂-K/R₂₋₄, wherein Xₙ is a plurality of any amino acid residues, T/S is an amino acid which is a threonine or a serine, P/X₁₋₂ is two amino acids at least one of which is a proline, and K/R₂₋₄ is two to four amino acids, at least two of which are lysine or arginine residues. Further “bipartite” or “dual” NLS amino acid sequences are described by the consensus sequence Xₙ-T/S-P/X₁₋₂-K/R₂₋₄-Xₙ-K/R₂₋₄ wherein Xₙ is any amino acid residues, T/S is an amino acid which is a threonine or a serine, P/X₁₋₂ is two amino acid residues at least one of which is a proline, and K/R₂₋₄ is two to four amino acid residues, at least two of which are lysine or arginine residues. Examples of naturally occurring bipartite NLS sequences are provided such as Adenovirus DBP NLS (SEQ ID NOs: 61 and 62), in which the dual basic regions are separated by at least about 40 residues, and human c-myc NLS (SEQ ID NOs: 48 and 49), in which the dual basic regions are separated by at least about 35 residues (see Table 1B).

Another aspect of the invention features phosphorylated peptides, for example, an amino acid sequence selected from the group consisting of:

| | |
|---|---|
| ADSQHSpTPPKKKR, | (SEQ ID NO: 12) |
| PPKKKRpTADSQHS, | (SEQ ID NO: 13) |
| PPKKKRTADSQHS, | (SEQ ID NO: 14) |
| ATADSQHSpTPPKKKRKV, | (SEQ ID NO: 15) |
| PPKKKRKVpTATADSQHS, | (SEQ ID NO: 16) |
| PPKKKRKVTATADSQHS, | (SEQ ID NO: 17) |
| RKVLGSSQNSSGSEASEpTPVKRRK, | (SEQ ID NO: 18) |
| SEASETPVKRRKGKRKVLGSSQNSS, | (SEQ ID NO: 19) |
| ADSQHSAPPKKKR, | (SEQ ID NO: 20) |
| ADSQHSTAPKKKR, | (SEQ ID NO: 21) |
| ADSQHSTPAKKKR, | (SEQ ID NO: 22) |
| ADSQHSTAAKKKR, | (SEQ ID NO: 23) |
| SQHSpTPPKK; | (SEQ ID NO: 25) |
| and | |
| PPKKpTSQHS. | (SEQ ID NO: 27) |

The abbreviation “pT” indicates a phosphorylated threonine residue. The threonine (T) can be substituted with a serine (S), or a phosphorylated serine, “pS”. Phosphorylated peptides can function as inhibitors of important biological targets, such as Pin 1, a proline isomerase. Such peptides are lead compounds in design of chemical derivatives capable of binding to DNA sequence targets. Also provided are designed variant peptides having a negatively charged residue instead of a threonine (or serine), for example, having an aspartic acid (D) or a glutamic acid (E) instead of a T or an S. These variant peptides, like the phosphorylated peptides, can function as inhibitors of important biological targets.

In another aspect, the invention provides any of the designed and synthesized isolated peptides herein as “polyvalent” peptides, which have enhanced binding to a target DNA molecule compared to the corresponding monovalent peptide. An isolated peptide of 13 amino acid residues in length, for example, ADSQHSTPPKKKR (SEQ ID NO: 3), can be synthesized in a divalent form, as a direct repeat of length 26 amino acids, (ADSQHSTPPKKKR)₂ or ADSQHSTPPKKKRADSQHSTPPKKKR, or can have a higher number of direct repeats (ADSQHSTPPKKKR)ₙ, or can have a fractional number repeat, for example, ADSQHSTPPKKKRADSQHST. Polyvalent NLS peptides contain direct consecutive repeats, or can contain repeats that separated by one or more “spacer” amino acid sequences.

In yet another aspect, the present invention provides an isolated peptide having an amino acid sequence of a nuclear localization signal (NLS), and the peptide binds to a protein target such as an enzyme such as a helicase. The peptide inhibits an ATPase activity of the helicase. For example, the helicase is a naturally occurring viral protein. Further, the helicase is involved in initiation of DNA replication. A different portion of the amino acid sequence of the peptide binds to each of the DNA target and to the protein target such as the helicase.

Also provided herein is a method of inhibiting DNA replication of a DNA virus, comprising contacting a nucleic acid sequence of a naturally occurring viral DNA molecule with a peptide as provided.

An aspect of the invention featured herein is a method of identifying a variant of a parent nucleotide sequence, the parent sequence encoding a parent peptide comprising an NLS sequence of amino acids, the derivative encoding a variant peptide having greater affinity for a target DNA nucleotide sequence than the parent peptide. The method involves displaying the parent peptide on a coat protein of a bacteriophage by inserting the parent nucleic acid sequence into the phage chromosome; mutagenizing the parent sequence in codons encoding residues of the peptide that can form a surface to bind to the DNA sequence, to produce a resulting library of variant peptides displayed on the phage coat. The method further involves adsorbing the library directly to and selectively eluting the library from an immobilized substrate comprising the target nucleotide sequence, such that peptides having greater affinity are eluted successively from the substrate, to obtain a phage clone displaying the variant peptide having greater affinity for the target DNA sequence than the parent peptide.

In an embodiment of this method, the peptide comprises at least about 8 amino acid residues to about 50 amino acid residues. Absorbing the library to the immobilized substrate is providing at least about 20 nM of peptide equivalents. In a further example of the method, the target DNA is a nucleotide sequence located at about an origin of replication of a virus, for example, the virus is a pathogen of a eukaryotic cell. The method can further include determining the nucleotide sequence encoding the derivative peptide, for example, batch sequencing of the nucleic acid of the encoding eluted phage, or cloning individual phage plagues and sequencing samples of a representative number of clones. The method can include chemically synthesizing the encoded amino acid sequence of the peptide carried by the phage.

An embodiment of the invention also provides a peptide produced by the above method, the peptide being related to a starting parental amino acid sequence which is genetically modified using phage display technology, to increase affinity of the parental peptide for a target DNA sequence. The invention provides a resulting library of the genetically modified peptides displayed on the phage coat, the resulting library being subjected to adsorption to and selective elution from an immobilized double-stranded DNA sequence of a target virus. The genetically modified peptide sequence displayed on the selected phage clone has greater affinity for the target DNA sequence of the target virus than the peptide having the starting amino acid sequence.

The peptides provided in the present invention can have at least one chemical modification, for example, the modification is a D-amino acid, or is a non-peptidic bond. Further, the modification is an amino acid substitution by a non-naturally occurring amino acid analogue. The modified peptide has greater binding affinity for the target DNA than the parent peptide. Also provided are the library of mutagenized peptides, and a variant of a peptide obtained by the above method.

Peptides provided using phage display technology can be selected by affinity methods, such as adsorption to and selective elution from a double-stranded DNA sequence of a virus. The phage clone displaying a selected genetically modified peptide sequence has greater affinity for the DNA sequence of the exemplary virus than the starting peptide. Affinity methods can also be used directly on a library of peptides derived from a parent sequence, which library can be adsorbed directly to a target DNA sequence immobilized on a column or a bead, the peptides then being eluted selectively so that those members of the library with greatest affinity are eluted into subsequent fractions rather than appearing in initial eluted fractions. A consensus sequence of peptides having greater affinity for the DNA target than the parent sequence can be determined by batch sequencing of the peptides. Exemplary substantially pure preparations of the peptide having different versions of the consensus can be synthesized and tested for affinity to the target DNA sequence.

In another aspect of the invention, methods of screening a chemical compound to identify an agent having ability to inhibit DNA replication of a DNA molecule are also provided herein. The method includes: providing a first reaction having the DNA molecule, the compound, and the substrates for DNA synthesis with a sample of an extract of eukaryotic cells, under conditions such that in a second reaction which is a control lacking the compound but is otherwise identical to the first reaction, the DNA molecule in the second reaction is replicated. The extract of eukaryotic cells, for example human cells, provides the DNA polymerase. The amount of DNA replication in the first reaction is compared with that in the second reaction, and is also compared with replication in a third reaction having a Cdk/NLS peptide rather than the compound and is otherwise identical to the first reaction. An amount of replication in the first reaction which is less than the amount in the second reaction indicates that the chemical is an inhibitor of DNA replication, and the amount of replication in the first reaction compared to that in the third reaction indicates the relative extent of inhibitory activity of the chemical compared to that of an inhibitory peptide.

Another aspect of the invention provided herein is a method of screening a chemical compound to identify an agent having ability to inhibit a helicase ATPase. The method involves a first reaction having the helicase, the compound, and a substrate for the ATPase activity, under conditions such that in a second reaction which is control lacking the compound but is otherwise identical to the first reaction, the helicase has ATPase activity. Then the amount of ATPase activity in each of the first and second reactions is compared with that in a third reaction having a Cdk/NLS peptide rather than of the compound and is otherwise identical to the first reaction. An amount of ATPase activity in the first reaction that is less than that in the second reaction indicates that the chemical is an agent which is a ATPase activity inhibitor, and the amount of ATPase activity in the first reaction compared to that the third reaction indicates the extent of inhibitory activity of the chemical compared to an inhibitory peptide.

In another aspect, invention features a method of inhibiting replication of a target DNA virus, by contacting the DNA of the virus with a peptide having an NLS (an amino acid sequence of a nuclear localization signal). The peptide binds to the DNA of the virus, such that replication of the virus is inhibited. For example, the peptide binds to a protein in the replication complex and inhibits multimerization of the protein, for example, inhibits formation of a multimeric active form of a helicase. Further, the peptide comprises about 10 to about 34 amino acid residues, for example, the peptide comprises about 13 to about 25 amino acid residues. Conditions under which the peptide binds to DNA include providing a peptide concentration of at least about 20 nM of the peptide, for example, providing a peptide concentration of at least about 50 nM of the peptide, providing a peptide concentration of at least about 500 nM of the peptide, or providing a peptide concentration of at least about 1,000 nM of the peptide.

The peptide preferably binds to a specific nucleotide sequence in the target virus DNA. For example, the specific nucleotide sequence in the DNA is located at about the origin of replication of the virus. Further, the step of delivering a peptide includes delivering a peptide that is not phosphorylated, in the event that the peptide comprises an amino acid residue selected from the group of serine, threonine and tyrosine. In general, the target DNA is double stranded. The step of delivering a peptide includes delivering a peptide having at least one chemical modification, for example, the amino acid analog is a D-amino acid, or the chemical modification is a non-peptide bond. The chemical modification is an amino acid substitution by an amino acid analogue. The peptide comprises at least one amino acid modification at a residue location to increase binding affinity. Further, prior to delivering the peptide, the method can comprise optimizing affinity of the peptide for the DNA of the target virus, by displaying a library of variant peptides on a filamentous bacteriophage, and selecting for increased affinity by binding the library to the DNA, and washing off phage of lesser affinity, and using buffers of increased stringency for selectively eluting those phage having increased affinity for the DNA.

In another embodiment, invention provides a method of obtaining a peptide capable of binding to a target DNA sequence, the method involving screening a library of diverse amino acid sequences. The method includes providing the library of peptides, each of the peptides having an amino acid sequence that is a variant of a highly basic parental amino acid sequence; and contacting the library of peptides with the target DNA sequence. The target DNA sequence corresponds to a sequence of a naturally-occurring nucleic acid obtained, for example, from a virus, or from a gene having aberrant expression that has been associated with a cancer. The library of peptides and target DNA are contacted under conditions for interacting; and members of the library that have bound to the target DNA sequence are selected. The target DNA sequence from virus can be from a pathogenic virus such as a papovavirus or a retrovirus, for example, the retrovirus is human immunodeficiency virus. A target DNA sequence from a genomic sequence associated with a cancer can be the HER-2/neu gene or the jun/fos AP1 gene, or another gene the overexpression of which is associated with a disease state such as an antoimmune disease, such as rheumatoid arthritis or multiple sclerosis.

Further, selecting members of the library that have bound to the target DNA is optionally a process selected from the group consisting of: binding to and eluting from the target DNA which was previously immobilized; precipitating magnetically the peptides bound to target DNA which was previously labeled with magnetic beads; and precipitating immulogically the peptides bound to the target DNA which was previously labeled with an antigenic material. Following the selecting of the peptides, the method further comprises eluting the selected peptides from the target DNA and analyzing the peptides by mass spectroscopy. Alternatively, the target DNA sequence is genetically engineered into a recombinant prey plasmid of a two-hybrid system, and providing a library of peptides is recombinantly inserting DNA encoding the library into a bait plasmid of a two-hybrid system. Further, the engineered bait plasmid is transformed into recipient cells already carrying the prey plasmid cells that receive successful high affinity-peptide encoding plasmids can be selected directly.

An aspect of the invention provides a method of inhibiting complete assembly of a multimeric DNA binding target protein in a target cell, the method comprising delivering to the cell a peptide having an amino acid sequence of a nuclear localization signal and, optionally, a site for phosphorylation; and providing conditions under which the peptide binds to the target DNA, such that complete assembly of the target protein is inhibited. The peptide comprises at least about 10 amino acid residues, for example, the peptide comprises at least about 13 amino acid residues. In related embodiments, the peptide comprises at least one amino acid modification at a residue in the amino acid sequence to increase binding affinity. Delivering the peptide in a related embodiment further comprises delivering a peptide that is optionally phosphorylated, e.g., the peptide is phosphorylated on an amino acid which is serine or threonine. Further, the phosphorylated peptide can be synthesized to contain the phosphate, or synthesized as a non-phosphorylated peptide, and altered enzymatically by adding a phosphate.

### BRIEF DESCRIPTION OF THE DRAWINGS

FIG. 1 is a set of photographs showing analysis of T-ag band shift reactions by gradient polyacrylamide gels electrophoresed in 0.5% Tris-borate-EDTA. Panel A shows reactions conducted in the presence of the 9-mer set of peptides. The 9-mer peptides are, from the top, SEQ ID NOs: 25-28. Panel B shows reactions conducted in presence of the 13-mer set of peptides, which are, from the top, SEQ ID NOs: 12, 3, 13 and 14. In each photograph, the reactions in lane 1 were conducted in the absence of either T-ag or peptide, and the reactions in lane 2 were conducted in the presence of T-ag (6 pmol) but in the absence of peptide. The reactions in lanes 3-6 were conducted in the presence of T-ag (6 pmol) and 20 nM of the indicated peptide. The positions of T-ag hexamers (H), double hexamers (DH) and “input DNA” are indicated.

FIG. 2 is a set of photographs showing analysis of band shift reactions by gradient polyacrylamide gels electrophoresed in 0.5% Tris-borate-EDTA. Reactions were conducted in the presence of the 17-mer set of peptides and 20 nM (panel A) of peptide or 10 nM (panel B) of peptide. The 17-mer peptides are, from the top, SEQ ID NOs: 15, 11, 16, and 17. As in FIG. 1, the reactions in lane 1 were conducted in the absence of either T-ag or peptide. The reactions in lane 2 were conducted in the presence of T-ag (6 pmol), but in the absence of peptide. The reactions in lanes 3-6 were conducted in the presence of T-ag (6 pmol) and 20 nM or 10 nM of the indicated peptide. The positions of T-ag hexamers (H), double hexamers (DH) and “input DNA” are indicated.

FIG. 3 is a bar graph that shows extent of peptide/DNA interactions by nitrocellulose filter binding assays. The interaction of the indicated peptide with a double stranded oligonucleotide containing the SV40 core origin (SEQ ID NO: 24) was detected by conventional nitrocellulose filter binding assays. All reactions were conducted under replication conditions in the presence of three different concentrations of peptide (0.5 mM, 1 mM and 2 mM). The percentage of input DNA bound to a given filter was determined by scintillation counting. As a control, the percentage of input DNA bound to a nitrocellulose filter in the absence of peptide was also determined. The 9-mer peptides are, from left to right, SEQ ID NO: 25, SEQ ID NO: 26, SEQ ID NO: 27, and SEQ ID NO: 28. The 13 mer peptides are, from left to right, SEQ ID NO: 12, SEQ ID NO: 3, SEQ ID NO: 13, and SEQ ID NO: 14. The 17-mer peptides are, from left to right, SEQ ID NO: 15, SEQ ID NO: 11, SEQ ID NO: 16, and SEQ ID NO: 17.

FIG. 4 is a set of photographs showing analysis of band shift reactions by gradient polyacrylamide gels electrophoresed in 0.5% Tris-borate-EDTA. Panel A is a photograph of reactions in the left panel conducted under replication conditions in the presence of a double stranded oligonucleotide containing the SV40 core origin. The reaction products formed in the presence of peptide 1₁₃(SEQ ID No: 12), peptide 2₁₃ (SEQ ID No: 3), peptide 3₁₃ (SEQ ID No: 13), and peptide 4₁₃ (SEQ ID No: 14) are shown in lanes 2-5, respectively in both panels. The reactions in Panel B were conducted in the presence of a control oligonucleotide termed the 64 bp enhancer control (SEQ ID No: 29; Joo, W. S., et al., 1998, Mol. Cell. Biol. 18:2677-2687). In both panels, the mobility of the input DNA is shown in lane 1, and the positions of the peptide/DNA complexes are indicated.

FIG. 5 is a photograph of results of a gel electrophoretic mobility shift of reactions conducted with the 9-mer set of peptides. The reactions were conducted under replication conditions in the presence of the 64 bp SV40 core origin (SEQ ID NO:24). The products of reactions formed in the absence of peptide are shown in lane 1. As a positive control, a peptide band shift was conducted with peptide 2₁₃ (SEQ ID No: 2; lane 3). Reactions products formed with the 9-mer set of peptides are shown in lanes 3-6. The positions of the input DNA and the peptide/DNA complexes are indicated.

FIG. 6 is a photograph of results of electrophoretic mobility shift assays conducted with peptide T124A. The products formed in reactions conducted under replication conditions in the presence of the SV40 core origin (SEQ ID NO: 24), but lacking peptide, are shown in lane 1. As additional controls, the products of reactions conducted in the presence of peptide 4-13 (SEQ ID No: 14), and peptide 2-13 (SEQ ID No: 2), are shown in lanes 2 and 3, respectively. The reaction products formed in the presence of peptide T124A₁₃ (SEQ ID No: 20) are shown in lane 4. An identical reaction, performed in the presence of the 64 bp enhancer control (SEQ I NO: 29), is shown in lane 6; the mobility of the 64 bp enhancer control in the absence of peptide is shown in lane 5.

FIG. 7 is a bar graph that shows results of filter binding assays for determining whether peptides derived from the NLS region of Bovine Papillomavirus E1 bind to DNA. Nitrocellulose filter binding assays were performed with the peptides indicated in the FIG. (P2-17 is SEQ ID NO: 11; BP-E1 is SEQ ID NO: 2; BP-E1 control is SEQ ID NO:19), and a double stranded oligonucleotide containing the SV40 core origin (SEQ ID NO:24). The nitrocellulose reactions performed with T-ag were conducted according to the DNA replication conditions described in the Examples section. In this series of experiments, two different concentrations of peptide (0.25 mM and 0.5 mM) were used. The percentage of input DNA bound to a given filter was determined by scintillation counting. “Control” indicates the percentage of input DNA bound to a nitrocellulose filter in the absence of peptide.

FIG. 8 is a line graph showing the results of SV40 in vitro replication reactions (pmol of nucleotide incorporation on the ordinate as a function of time, in min on the abscissa) conducted in the presence of the NLS based peptides. Reactions were conducted in 30 μl final volume using conditions as described in the Examples section. Peptides showing high affinity for DNA as tested herein were further tested for ability to inhibit SV40 in vitro replication. To each reaction, a sample of 1 μl, 2 μl or 4 μl volume of the indicated peptide was added (corresponding to addition of 10 nM, 20 nM or 40 nm of peptide). At the indicated times, aliquots were assayed for acid-insoluble radioactivity. The reaction components present in the individual experiments are indicated to the right of the Figure. Control peptides are designated by (C). SEQ ID NOs for the sequences listed in the Figure: E1 is SEQ ID NO: 2, E1C is SEQ ID NO: 19, and P2-17 is SEQ ID NO: 11. The designations “+T” and “−T” indicate the presence and absence of T-ag, respectively, with no additional peptide inhibitor present in the reaction.

FIG. 9 Panel A shows the amino acid sequences of peptides as listed from top to bottom in SEQ ID NOs: 12, 3, 13, 14, 15, 11, 16 and 17, respectively, centered on Thr124, in the amino acid sequence of SV40 T-ag; the subscripts in the peptide number denote the size of the peptide. The peptide numbering system is based on the corresponding residues in full-length T-ag. Residues that are part of the T-ag NLS are shown in bold. The underlined amino acid residues in the 13-mer wild type peptides (SEQ ID NOs: 12 and 3) indicate the recognition motif for cyclin/Cdk kinase.

FIG. 9 Panel B is a photograph of a gel electrophoretogram showing results of T-ag band-shift reactions conducted in the presence of the “1 3-mer set” of peptides and the 64-bp core oligonucleotide (SEQ ID NO: 24). Control reactions in lane 1 were conducted in the absence of either T-ag or peptide. The reaction in lane 2 was conducted in the presence of T-ag (6 pmol). The reactions in lanes 3-6 were conducted in the presence of T-ag (6 pmols) and 20 nM of the indicated peptide (SEQ ID NOs: 12, 3, 13, and 14, respectively). The positions of T-ag hexamers (H) and double hexamers (DH) are indicated.

FIG. 9 Panel C is a bar graph showing detection and quantitation of peptide/DNA interactions by nitrocellulose filterbinding assays. All reactions were conducted under conditions for replication in the presence of three different concentrations of peptide (0.5 mM, 1 mM and 2 mM), employing a 64-bp double stranded oligonucleotide containing the SV40 core origin (SEQ ID NO: 24). The percentage of input DNA bound to a given filter was determined by scintillation counting. As a control, the percentage of input DNA bound to a nitrocellulose filter, in the absence of peptide, was also determined (control). The peptides are, respectively: “9-mers” are SEQ ID NOs: 25, 26, 27, and 28; “13-mers” are SEQ ID NOs: 12, 3, 13, and 14; and “17-mers” are SEQ ID NOs: 15, 11, 16, and 17.

FIG. 9 Panel D shows two electrophoretograms with the results of band shift reactions conducted with the 13-mer set of peptides (SEQ ID NOs: 12, 3, 13 and 14). The reactions in the left panel were conducted under replication conditions in the presence of a double stranded oligonucleotide containing the SV40 core origin (SEQ ID NO: 24). The mobility of the input DNA is indicated in lane 1. The reaction products formed in the presence of peptide T-1₁₃, peptide T-2₁₃, peptide T-3₁₃ and peptide T-4₁₃, SEQ ID NOs: 12, 3, 13 and 14, respectively, are presented in lanes 2-5, respectively. The reactions in the right panel were conducted in the presence of a 64-bp non-origin containing oligonucleotide termed the “enhancer control” (SEQ ID NO: 29; Joo, W. S. et al. 1998, Mol. Cell. Biol. 18:2677-2687). The mobility of the input DNA is shown in lane 1; the reaction products formed in the presence of peptide T-1₁₃ (SEQ ID NO: 12), peptide T-2₁₃ (SEQ ID NO: 3), peptide T-3₁₃ (SEQ ID NO: 13) and peptide T-4₁₃ (SEQ ID NO: 14) are presented in lanes 2-5, respectively. The positions of the peptide/DNA complexes are indicated in both Figure s.

FIG. 10 Panel A shows peptides derived from Bovine Papillomavirus E1 in the vicinity of the NLS element. Peptide numbering is based on the system used to designate residues in Bovine papillomavirus E1 protein (SEQ ID NOs: 18, 2 and control SEQ ID NO: 19). Peptides E1-P1₂₅ (SEQ ID NO: 18) and E1-P2₂₅ (SEQ ID NO: 2) both contain a bipartite NLS found in Bovine papillomavirus E1; they differ in that peptide E1-P1₂₅ (SEQ ID NO: 18) contains a phosphate on Thr102. The control peptide E1-4₂₅ (SEQ ID NO: 19) was designed by swapping the residues normally found on each side of glycine 96. Residues in bold show the bipartite Bovine papillomavirus E1 NLS, and underlined residues represent a putative recognition motif for the cyclin/Cdk kinase (Lentz, M. R. et al. 1993. J. Virol. 67: 1414-1423).

FIG. 10 Panel B is a photograph of an electrophoretogram showing results of T-ag band shift reactions conducted in the presence of the Bovine papillomavirus E1 “Cdk/NLS” set of peptides. The control reactions in lane 1 were conducted in the absence of either T-ag or peptide. The reaction in lane 2 was conducted in the presence of T-ag (6 pmol). The reactions in lanes 3-5 were conducted in the presence of T-ag and 5 nmol of the peptide having the amino acid sequence and SEQ ID NO as indicated in the Figure and in Panel A (final concentration of 0.25 mM).

FIG. 10 Panel C is a bar graph showing quantities that bind to DNA of each of the peptides derived from the “Cdk/NLS” region of Bovine papillomavirus E1. Nitrocellulose filter binding assays, conducted under replication conditions, were performed in the presence of each of the peptides indicated and the 64-bp SV40 core origin containing oligonucleotide (SEQ ID NO: 24). In this series of experiments, two different concentrations of peptide (0.25 mM and 0.5 mM) indicated in the Figure and identified in Panel A were used. The percentage of input DNA bound to a given filter was determined by scintillation counting. As a control, the percentage of input DNA bound to a nitrocellulose filter in the absence of peptide was also determined.

FIG. 11 is a set of Figures showing sequences and results using variant versions of the wild type SV40 T-ag 13 amino acid peptide (as shown in SEQ ID NO:3), in order to identify those residues necessary for DNA binding and for regulation of this process.

Panel A shows the sequences of the “mutant set” of peptides formed by substituting alanine (A; SEQ ID NOs 20-23, respectively) or aspartic acid (D; SEQ ID NO: 73) for residue T124 as indicated in the left hand column, with the sequences obtained shown in the right hand column. In the P125A/P126A₁₃ double mutant (SEQ ID NO: 23), alanine residues replaced both prolines at positions 125 and 126. In the K128A mutant, an alanine replaces lysine at position 128 (SEQ ID NO: 74).

Panel B is a photograph of an electrophoretogram analysis determining whether the “mutant set” of peptides disrupted T-ag oligomerization on a 64-bp oligonucleotide (SEQ ID NO: 24) DNA containing the SV40 core origin. The locations of T-ag hexamers (H) and double hexamers (DH) are indicated. The reaction analyzed in lane 1 was conducted in the absence of protein, and the reaction in lanes 2-10 were performed in the presence of T-ag (6 pmol). The control reaction in lane 2 shows H and DH in the absence of any inhibitory peptide. The reaction in lane 3 was conducted with phosphorylated peptide (SEQ ID NO; 12), and the reaction in lane 4 with unphosphorylated peptide (SEQ ID NO: 3), respectively. Lane 3 shows no inhibition of oligomerization of T-ag, i.e., H and DH were observed, and lane 4 shows complete inhibition of oligomerization. The reaction in lane 5 was conducted with the T124A mutant (T124A₁₃; SEQ ID NO: 20) and shows that this mutation does not destroy the inhibitory property of the peptide, i.e., that a T at 124 is not required for inhibition of oligomerization. The reactions in lane 6 was conducted in the presence of the T124D mutant peptide (SEQ ID NO: 73), and shows that this mutation abolishes inhibition of oligomerization. The reactions in lanes 7-9 were conducted in the presence of each of peptides P125A₁₃ (SEQ ID NO: 21), P126A₁₃ (SEQ ID NO: 22), or the P125A/P126A₁₃ double mutant (SEQ ID NO: 23), respectively. Full inhibition of oligomerization is observed. The reaction in lane 10 was conducted in the presence of K128A mutant peptide (SEQ ID NO: 74), and shows that mutation of the NLS residue K to a non-basic A abolished inhibition of oligomerization. Panel C is a graph showing quantitation of interactions between the mutant set of peptides and DNA by nitrocellulose filter binding assays. The reactions contained the 64-bp SV40 core origin oligonucleotide and 20 nmole (1 mM final concentration) of each peptide as indicated. The percentage of input DNA bound to a given filter was determined by scintillation counting. About 60%-80% of DNA was bound by peptides that were non-phosphorylated, or mutated to have A in place of T at 124, P at 125, P at 126, or both P at 125 and 126. D at 124 in place of T, or A at 128 in place of K abolished ability to bind to DNA in this assay.

Panel D is a photograph showing results of electrophoretic mobility shift assays performed with peptide T124A₁₃ (SEQ ID NO: 20). The products formed in reactions shown in lanes 1-4 contained the SV40 core origin (SEQ ID No: 24), and in lanes 5-6 contained the enhancer control 64 bp DNA (SEQ ID No: 29). A reaction lacking peptide is shown in lane 1, and indicates mobility of input DNA. As additional controls, the products of reactions conducted in the presence of control peptide T-4₁₃ having a rearranged amino acid sequence (SEQ ID NO: 14; lane 2), and in the peptide of wild type sequence T-2₁₃ (SEQ ID NO: 3; lane 3) are shown. The reaction products formed in the presence of peptide T124A₁₃ (SEQ ID NO: 20) is shown in lane 4. An identical reaction, performed in the presence of peptide T124A₁₃ (SEQ ID NO: 20) and the 64-bp enhancer control (SEQ ID NO: 29), is shown in lane 6; the mobility of the 64-bp enhancer control in the absence of peptide is shown in lane 5. Mobility shifts are seen with wild type sequence peptide (lane 3), and T124A peptide (lanes 4 and 6), but not with a control peptide having a rearranged sequence (lane 2).

FIG. 12 is a drawing of a model depicting the phosphate regulated assembly of the second T-ag double hexamer of sub-fragments of the core origin. The small circle represents the T-ag-origin binding domain while the larger oval represents the remaining residues in T-ag. For simplicity, single T-ag monomers represent T-ag hexamers. Panel A shows the formation of the first hexamer, which is independent of phosphorylation status of Thr124. As shown herein, an unphosphorylated “Cdk/NLS” motif in T-ag, indicated as a loop outside of the small circle, binds to DNA, an event that blocks formation of the second hexamer. Panel B shows phosphorylation of Thr124. As shown by the examples herein, and indicated by the loop being drawn back into the small circle in Panel B, the “Cdk/NLS” motif no longer binds to DNA. Panel C shows that as the Cdk/NLS motif is removed from DNA, the protein/protein and protein/DNA interactions necessary for double hexamer formation take place.

FIG. 13 is an autoradiograph with results of an assay of ATP hydrolysis by T-ag helicase activity, showing the effects of the presence of peptides on the ATPase activity. ATP hydrolysis (of ³²P-ATP) in samples was assayed by incubating with T-ag, crosslinking with glutaraldehyde, removing unincorporated radioactive nucleotides with Bio-Spin P-30 columns, drying the eluate in a Speedvac, resuspending the samples in formic acid, and chromatographing the samples on PEI TLC plates. Lanes 2 and 3 are duplicate positive controls, with no added peptide. Lanes 4 and 5 are duplicate tests of phosphorylated peptide T-1₁₋₁₇ (SEQ ID NO: 15); lanes 6 and 7 are duplicate tests of unphosphorylated peptide T-2₁₃ (SEQ ID NO: 3); lanes 8 and 9 are duplicate tests of unphosphorylated peptide E1-P2₂₅ (SEQ ID NO: 2); and lanes 10 and 11 are duplicate tests of phosphorylated peptide E1-P1₂₅ (SEQ ID NO: 18). In the presence of a phosphorylated peptide of the sequences indicated herein, the T-ag ATPase functions to bind ATP and convert it to ADP. The data in the Figure show that unphosphorylated peptides inhibit this reaction.

### DETAILED DESCRIPTION OF EMBODIMENTS

Understanding the mechanism by which peptides derived from T-ag to bind to DNA, and the nature of the peptides that bind, can provide anti-viral agents, and agents that block eukaryotic gene transcription, for example of a gene related to a cancer, and methods of use of these agents.

Classes of peptides that are known to bind to DNA include: Zn finger-containing peptides (Kim, J. S. et al., 1998, Proc. Natl. Acad. Sci. USA 95:28 12-2817), and a helix containing peptides (Chin, J. W. et al., 2001, J. Am, Chem. Soc. 123:2929-2930). However the small sizes of the nucleotide sequences recognized by Zn finger and a helix peptides, 3 and 5 base pairs, respectively (Chin, J. W. et al., 2001, J. Am. Chem. Soc. 123:2929-2930), and the relative sizes and complexity of the amino acid sequences involved in DNA binding indicate that these proteins are not optimal agents to function in a regulatory capacity.

As used herein and in the claims, the following terms shall have the following meanings, unless the context otherwise requires.

“Helicase” means an enzyme that unwinds a localized region of DNA consuming ATP (the helicase having an ATPase activity). Once assembled on the core origin, T-ag is converted into a 3′ to 5′ DNA helicase (Stahl, H., et al., 1986, EMBO J. 5:1939-1944) that is able to unwind the SV40 origin and thereby establish two replication forks (Bullock, P. A., et al., 1989, Proc. Natl. Acad. Sci. USA 86:3944-3948). Like SV40, many other DNA viruses (e.g., papillomaviruses) encode initiator proteins that locate their respective origins, and upon protein assembly, are functional helicases.

“Papovavirus” means a family of viruses that infect animal cells, including papillomaviruses and polyoma viruses. Papilloma viruses include human papilloma viruses including more pathogenic strains such as strains 16 and 18, and Bovine Papillomavirus. Polyoma viruses include Simian Virus 40 (SV40) virus.

“Substantially pure” means that a composition comprises at least 75%, for example at least 85%, or at least 95%, or at least 98% of that component on a weight to weight basis, or if in solution, on a weight to volume basis.

“Subject” shall mean, without limitation, a mammal such as a human, ape, monkey, horse, cow, sheep, goat, pig, rodent such as rat or mouse, cat, dog, or other mammal. A subject may be asymptomatic, or may be a patient having a viral infection or a cancer.

“Peptide” shall mean a polypeptide or oligopeptide comprising primarily amino acids polymerized by peptide bonds, for example from 5 to 50 amino acids in length. Peptides herein are preferably at least about 5 amino acids in length, for example, are at least about 10 amino acids in length, for example, are about 11, 12, or 13 amino acids in length.

The term “Cdk” phosphorylation site is generally known to be S/T-Pro-X-Z, where S/T is either a serine or a threonine, X is a polar amino acid, and Z is a basic amino acid (Moreno, S. et al. 1990 Cell 61: 549-551)

The term “derivative” of an amino acid means a non-naturally occurring chemically related form of that amino acid having an additional substituent, for example, an N-carboxyanhydride group, a γ-benzyl group, an ε,N-trifluoroacetyl group, or a halide group attached to an atom of the amino acid. A peptide derivative may contain at least one non-peptidic bond, for example, a phosphate bond or a phosphorothioate bond, between two adjacent amino acids. A peptide derivative may contain one or more non-naturally occurring amino acids.

Many derivatives of synthetic peptides having increased pharmacological life in vivo have been synthesized. Pharmacological life is affected by the fact that peptides may be digested by amino- or carboxy- peptidases in serum or in other biological fluids. Therefore, proteolysis of the peptides may effectively remove the peptides from the subject (Bennett, K., et al., 1992, Eur. J. Immunol. 22:1519). To reduce or eliminate potential proteolysis, modification of the peptides, for example, N-methylation of backbone nitrogens in the peptides, which are not involved in essential hydrogen bonding interactions, could produce a peptide derivative that is resistant to proteolysis (Falconi, F., et al., 1999, Nature Biotechnology 17:562). In Falconi et al., N-methylation of a hemagglutinin (HA) peptide to produce a modified peptide derivative yielded a compound that was substantially less sensitive to digestion by cathepsin B. The resulting protease resistant peptide can also be a substantially better inhibitor of T-antigen double hexamer formation and binding to DNA. Cyclic forms of the NLS-containing peptides herein are also included among various derivatives that can be synthesized.

In one embodiment, the invention provides derivatives of synthetic peptides having a chemical alteration in one or both of the peptide backbone or the amino acid side chains. These derivatives can have increased binding affinity to DNA and increased inhibitory activity and/or resistance to proteolysis. Alanine (A) can be substituted with one or more conformationally restricted aromatic compounds, Tic, which is tetrahydroisoquinoline-(S)-3-carboxylic acid), Thiq, which is tetrahydroisoquinoline-(S)-1-carboxylic acid), and Disc, which is (dihydroisoindole-(S)-2-carboxylic acid), and the blocked Cys compounds C(Acm),which is acetamido-methyl-Cys, C(Prm), which is propylamidomethyl-Cys, and C(Ace), which is acetyl-Cys. Furthermore, MePhg, which is methylphenyl-Gly, and Nva, which is norvaline, provided increased binding affinity. Substitution by some of the peptidomimetics can result in improved inhibition of gene expression.

A peptide may be produced by chemical synthesis, for example, by a polymerization reaction in solution, or using solid state methods, for example, by an automated peptide synthesizer. Peptides can also be produced biologically, for example, by cellular synthesis from an encoding nucleic acid, for example attached as a fusion to a larger protein, and isolated from culture medium or from an intracellular compartment. Peptides attached as a fusion to a larger protein may be cleaved enzymatically or chemically from the larger protein.

The terms “9-mer” and “13-mer” refer to peptides of 9 amino acids and 13 amino acids in length, comprising a unique sequence of amino acids indicated by the SEQ ID NO shown. The term “polyvalent” refers to a peptide of unique sequence synthesized as a longer sequence of amino acids which comprises one or more integral or partial repeats, without limitation as to orientation, of the unique sequence. Polyvalent peptides, consisting for example of two or three iterations of a peptide of known sequence, are referred to as “dimers” or “trimers”, respectively, as shall be understood from the context.

The term “basic” amino acid means amino acids, histidine (H or his), arginine (R or arg) and lysine (K or lys), which confer a positive charge at physiological values of pH in aqueous solutions on peptides containing these residues.

The term “NLS” site (or nuclear localization site) as used herein refers to a highly basic amino acid sequence the presence of which in an amino acid sequence of a peptide or a protein directs entry of the peptide or protein into the nucleus of a cell, compared to that of a peptide or a protein lacking that amino acid sequence.

The term “analog” means a non-naturally occurring non-identical but chemically related form of the reference amino acid. For example, the analog can have a different steric configuration, such as an isomer of an amino acid having a D-configuration rather than an L-configuration, or an organic molecule with the approximate size and shape of the amino acid, or an amino acid with modification to the atoms that are involved in the peptide bond, so as to be protease resistant when polymerized in the context of a peptide or polypeptide.

“Increased affinity” of a variant peptide in comparison to a parental peptide shall mean, for example, 1.5-fold more affinity, or 2 fold, or 4 fold, or 10 fold, 20 fold, or 50 fold greater affinity for a nucleic acid than that of a parental peptide.

“Library” of peptides, or a library of bacteriophage displaying peptides, means a plurality of peptides, or phage bearing peptides, respectively, each peptide member of the library bearing an amino acid sequence, the sequences being related to a parental sequence of amino acids. While a library of peptides may be displayed on a surface protein of a phage, peptide libraries may be constructed chemically and tested as arrays (macroarrays and microarrays, reviewed by Reimer, U. et al., 2002, Current Opinion in Biotechnol. 13: 315-320) and custom synthesis of a library of peptides based on an amino acid sequence is commercially available (for example, from The Peptide Laboratory, Benicia, Calif.; from the Tufts University Core Facility, Boston Mass.; or from a similar facility).

“Conditions suitable for peptide binding” comprise peptide concentrations from about 20 nM to about 1000 nM (micromolar), for example, at least 20 nM, at least 50 nM, at least 100 nM, at least 200 nM, at least 500 nM.

“Phosphorylated” peptides have been produced to contain a phosphate group added to the hydroxyl of a serine or threonine residue in the amino acid sequence of the peptide. Unless designated as phosphorylated, the peptides described herein are non-phosphorylated.

“Buffers of increased stringency” as described herein refer specifically to a set of buffers each otherwise identical and containing a property to a greater extent than the previous buffer, the property causing dissociation of a peptide from a nucleic acid. Examples include: buffers having increasing concentrations of a detergent; buffers having increased concentration of a chaotropic agent, buffers having decreasing pH; buffers of increasing temperature; and buffers having increased concentration of a salt.

Characteristics of T-Antigen Proteins

T-ag binds to several cellular replication factors, including pol α-primase complex (Gannan, J. B., et al., 1987, Nature (London) 329:456-458; and Smale, S. T., et al., 1986, Mol.Cell Biol. 6:4077-4087), human single stranded binding protein (HSSB/RPA; Weisshart, K., et al., 1998, J.Virol. 72:9771-9781) and topoisomerase I (Simmons, D. T., et al., 1996, Virol. 222:365-374).

A region of T-ag termed the T-ag-obd (origin binding domain) is capable of independently locating to the central region of the core origin (Kim, H. Y., et al., 1999, J.Virol. 73:7543-7555). An important function of the T-ag-obd is site-specific binding to individual GAGGC pentanucleotides within the core origin. The limits of the T-ag-obd were identified as residues 131-260 (T-ag-obd ₁₃₁₋₂₆₀; reviewed in Fanning, E., et al., 1992, Ann. Rev. of Biochem. 61:55-85). Mutagenesis studies located many of the important sub-regions in this domain, for example, the residues involved in binding to the origin (Wun-Kim, K., et al., 1993, J. Virol. 67:7608-7611). The solution structure of the T-ag-obd₁₃₁₋₂₆₀ was determined by nuclear magnetic resonance spectroscopy (Luo, X., et al., 1996, Nature Struct. Biol. 3:1034-1039). The two regions of the T-ag-obd that interact with individual pentanucleotides (Wun-Kim, K., et al., 1993, J.Virol 67:7608-7611) define a continuous surface on the protein.

The crystal structure of papillomavirus E1 protein DNA binding domain has been determined (Enemark, E. J., et al., 2000, Molec. Cell 6:149-158). Despite a sequence identity of only 6%, the three dimensional structure of the E1 DNA binding domain is nearly identical to that of the T-ag-obd (RMSD is 2.4 Å).

The T-ag-obd₁₃₁₋₂₆₀ is a monomer in solution (Luo, X., et al., 1996, Nature Struct. Biol. 15 3:1034-1039). When it binds to the SV40 core origin, it interacts exclusively with site II (Kim, H. Y., et al., 1999, J. Virol. 73:7543-7555). Related studies indicate that it binds to site 11 as a dimer and preferentially interacts with pentanucleotides 1 and 3 (Joo, W. S., et al., 1997, J. Virol. 71:3972-3985). However, when pentanucleotides 1 and 3 are not present, the T-ag-obd₁₃₁₋₂₆₀ can also interact with pentanucleotides 2 and 4. Why the T-ag-obd₁₃₁₋₂₆₀ cannot simultaneously bind to all four pentanucleotides is not understood, although it has been speculated that binding of T-ag-obd₁₃₁₋₂₆₀ to a given pentanucleotide obscures access for binding to neighboring pentanucleotides.

Studies employing electron microscopy techniques have provided additional insights into T-ag oligomerization and assembly on the core origin. T-ag assembles as a bi-lobed structure on the core origin, each lobe containing 6 monomers of T-ag (Dodson, M., et al., 1987, Science 238:964-967). Since preformed hexamers cannot interact with the SV40 origin, it is believed that T-ag binds DNA as a monomer and subsequently assembles into hexamers (Dean, F. B., et al., 1992, Journal of Biological Chemistry 267:14129-14137).

SV40 DNA replication takes place during the “S phase” of the cell cycle (Pages, J., et al., 1973, J. Virol 12:99-107). Activation and deactivation of proteins via phosphorylation and dephosphorylation are important determinants of progression through the stages in the cell cycle. Cyclin/Cdk complexes are necessary for SV40 replication (D'Urso, G., et al., 1990, Science 250:786-791). Further, cell cycle dependent phosphorylation of T-ag at residue Thr124 is essential for initiation of SV40 replication (Moarefi, 1. F., et al., 1993, J. Virol. 67:4992-5002).

A mutant T-ag protein containing a Thr to Ala substitution at position 124 (abbreviated T124A; Schneider, J., et al., 1988, J. Virol 62:1598-1605), has been used in studies designed to understand how Thr124 phosphorylation controls initiation of viral replication. T124A molecules have helicase activity, assemble into both hexamers and double hexamers on the core origin, distort the structure of the core origin, bind to cellular proteins required for initiation, and yet they do not support DNA unwinding or DNA synthesis (Weisshart, K., et al., 1999, J. Virol. 73:2201-2211).

Assembly of the first T-ag hexamer does not depend on the phosphorylation status of Thr124 (Barbaro, B. A., et al., 2000, J. Virol. 74:8601-8613). However, recent studies indicate that formation of the second hexamer is regulated, via an unknown mechanism, by phosphorylation of Thr124 (Barbaro, B. A., et al., 2000, J. Virol. 74:8601-8613; Weisshart, K., et al., 1999, J Virol. 73:2201-2211). Regions of T-ag flanking Thr124 contain a number of amino acid residues involved in related regulatory events. For instance, T-ag residues 126-132 contain the nuclear localization sequence (NLS) used for nuclear translocation (Jans, D., et al., 1998, Medicinal Res. Rev. 18:189-223), and residues 124 to 127 comprise the recognition motif for the cyclin/cylin dependent kinase motif (Cdk). The mechanism whereby phosphorylation of Thr124 regulates the assembly of the second hexamer has been heretofore unknown.

To explore how phosphorylation of Thr124 regulates the assembly of the second hexamer, a series of peptides derived from the region surrounding Thr124 was synthesized. These peptides were first tested for their ability to inhibit well characterized initiation events, such as T-ag assembly. Data herein show that small NLS containing peptides bind to DNA in a phosphate dependent manner.

To establish if these observations are unique to peptides derived from the T-ag NLS, a set of peptides containing the NLS region of Bovine Papillomavirus E1 were also synthesized. Data herein demonstrate that peptides based on the E1 NLS also bind to DNA. Moreover, the bi-partite NLS derived from Bovine Papillomavirus is able to block DNA replication in vitro.

A 13 amino acid peptide centered on SV40 T-ag Thr124 is provided herein, and this peptide is shown to bind to DNA. This is a surprising result, as previous data have identified the DNA binding domain of SV40 to a region of SV40 T-ag protein spanning residues 131-249 (Joo, W. S. et al., 1997, J.Virol. 71:3972-3985). Further, if Thr124 was phosphorylated, the peptide was found herein not to bind to DNA. These observations indicate that: an additional region of T-ag binds to DNA, and that a critical component of the cell cycle switch also regulates the initiation of SV40 DNA replication.

Double hexamer formation on individual assembly units is shown to be dependent upon the phosphorylation status of Thr124. In contrast, single hexamer formation is independent of the phosphorylation status of Thr124. These results indicate that completion of T-ag oligomerization is directly controlled by cyclin/Cdk phosphorylation, and that the cell cycle machinery controls T-ag oligomerization and thus initiation of DNA replication.

Disease Considerations

It has been estimated that viruses, including papillomaviruses types 16, 18, and hepatitis B, cause 15% of all human cancers, particularly in developing countries (Trichopoulos, D., et al., 1996, Scient. Amer.:80-87). Therefore, it is important to understand the mechanism of viral DNA replication, and how this process is controlled.

The DNA sequence features of the SV40 core origin are very similar to the sequences present in the core origins of other human (BK and JC viruses) and murine (polyomavirus) papovaviruses (Li, L., et al., 1995, J. Virol. 69:7570-7578). Similarities between the SV40 origin and the bovine papillomavirus origin of replication have been noted (Chen, O., et al., 1998, J. Virol. 72:2 567-2576). Moreover, “initiator” molecules encoded by a number of papovaviruses are highly homologous (Simmons, D. T., et al., 1990, J. Virol 64:4858-4865).

Table 1A lists NLS sites of exemplary viruses. Additional NLS sequences are listed in Table 1B, and are described in J. Garcia-Bustos et al., 1991 Biochim. Biophys. Acta 1071: 83-101, incorporated herein by reference.

TABLE 1A

Sequences of papovavirus
T-antigens at Cdk/NLS sites

| | |
|---|---|
| Vi- | SEQ |

| | | | | |
|---|---|---|---|---|
| rus | Host | Residues | Sequence | ID NO |

| | | | | |
|---|---|---|---|---|
| SV40 | Rhesus Monkey | 118-130 | ADSQHSTPPKKKR | 3 |
| BKV | Human | 120-132 | ADSQHSTPPKKKR | (3) |
| JCV | Human | 120-131 | G - SQHSTPPKKKR | 6 |
| LPV | African Monkey | 181-193 | QSSYTCTPPKRKK | 7 |
| HaPV | Hamster | 243-255 | QQSHHNTTPKKPP | 8 |
| PyV | Murine | 272-284 | QSSFNATPPKKAR | 9 |
| KV | Murine | 134-146 | PARSQATPPKKKA | 10 |

TABLE 1B
Additional nuclear localization signals (NLS)

SEQ

| | |
|---|---|
| Deduced signal | ID |

| | | | |
|---|---|---|---|
| Source | Nuclear protein | sequence^{(C-terminal position)} | NO |

| | | | |
|---|---|---|---|
| Yeast | MATα2 | KIPIK⁷ | 31 |
| Yeast | MATα2 | VRILESWFAKNI¹⁵² | 32 |
| Influenza | Nucleoprotein | AAFEDLRVRS³⁴⁵ | 33 |
| virus | | | |
| Yeast | rProtein L3 | PRKR²¹ | 34 |
| Polyoma | Large T | VSRKRPRPA¹⁹⁷ | 35 |
| virus | | PKKARED²⁸⁶ | 36 |
| SV40 | Vp1 | APTKRK⁶ | 37 |
| Adenovirus | Ela | KRPRP²⁸⁹ | 38 |
| Frog | Nucleoplasmin | RPAATKKAGQAKKKKLD¹⁷² | 39 |
| Rat | Glucocorticoid | KKKLK⁵¹⁷ | 40 |
| | receptor | | |
| Monkey | v-sis (PDGF B) | RVTIRTVRVRRPPKGKHRK²⁵⁵ | 41 |
| Yeast | Histone 2B | GKKRSKA³⁵ | 42 |
| Chicken | v-rel | KAKRQR³⁰³ | 43 |
| Influenza | NS1 | DRLRR³⁶ | 44 |
| virus | | PKQKRK²²¹ | 45 |
| Frog | N1 | VRKKRKT⁵³⁷ | 46 |
| | | AKKSKQE⁵⁵⁴ | 47 |
| Human | c-myc | PAAKRVKLD³²⁸ | 48 |
| | | RQRRNELKRSF³⁷⁴ | 49 |
| Human | lamin A | TKKRKLE⁴²² | 50 |
| HTLV-I | Rex (p27^{x-111}) | PKTRRRP⁸ | 51 |
| | | SQRKRPP¹⁷ | 52 |
| Adenovirus | pTP | RLPVRRRRRRVP³⁷³ | 53 |
| HIV-1 | Tat | GRKKR⁵² | 54 |
| Frog | Lamin L₁ | VRTTKGKRKRIDV⁴²¹ | 55 |
| Rabbit | Progesterone | RKFKK | 56 |
| | receptor | | |
| HIV-1 | Rev | RRNRRRRW⁴⁵ | 57 |
| Yeast | SKI3 | IKYFKKFPK³¹⁴ | 58 |
| Human | PDGF A-chain | PRESGKKRKRKRLKPT²¹¹ | 59 |
| Mouse | c-abl | KKKKK⁶²⁸ | 60 |
| Adenovirus | DBP | PPKKR⁴⁶ | 61 |
| | | PKKKKK⁸⁹ | 62 |
| Chicken | c-erb-A | SKRVAKRKL¹³⁵ | 63 |
| Human | c-myb | PLLKKIKQ³²⁸ | 64 |
| Human | N-myc | PPQKKIK-S³⁴⁴ | 65 |
| Human | p53 | PQPKKKP³²² | 66 |
| Human | Hsp70 | FKRKHKKDISQNKRAVRR²⁶⁷ | 67 |
| Hepatitis B | Core protein | SKCLGWLWG²⁹ | 68 |
| virus | | | |
| Chicken | Etsl | GKRKNKPK³⁸³ | 69 |
| Yeast | rProtein L29 | KTRKHRG¹² | 70 |
| | | KHRKHPG²⁹ | 71 |
| SV40 | VP2(VP3) | PNKKKRK | 72 |

NLS sequences are usually identified on the basis of homology with another NLS and therefore do not necessarily indicate NLS functionality (J. Garcia-Bustos et al., 1991 Biochim. Biophys. Acta 1071: 83-101).

Understanding the molecular details of T-ag assembly on the SV40 core origin, and regulation of this process, can provide insights into details of similar protein/origin interactions that occur in other DNA viruses. Further, elucidating the components steps and mechanisms of regulation of initiation and synthesis of viral DNA replication provides targets for the development of therapeutic agents that can control of these viruses and associated disease states. For example, JC virus is associated with the human demyelinating disorder progressive multifocal leukoencephalopathy (PML), a disease with a relatively high incidence (about 5%) in AIDS patients (White, F., et al., 1992, J. Virol. 66:5726-5734). JC virus is also associated with tumors of neural origin, including medulloblastomas, glioblastomas, neuroblastomas and meningiomas (reviewed in Small, J. A., et al., 1986, Proc. Nati. Acad. Sci. USA 83:8288-8292).

Further, SV40 T-ag is a eukaryotic DNA helicase (Stahl, H., et al., 1986, EMBO J. 5:1939-1944). Given that an increasing number of helicase-related diseases have been identified (e.g., Ellis, N. A., et al., 1995, Cell 83:655-666; Epstein, C. J., et al., 1996, BioEssays 18:1025-1027; Friedberg, E. C., 1992, Cell 71:887-889; and Lombard, D. B., et al., 1996, Trends Genet. 12:283-286), it is important to understand the assembly and regulation of this class of enzymes.

Characterization of Interactions of T-ag and the T-ag-obd with the SV40 Origin.

Purification of SV40 T-ag DNA-binding domain T-ag-obd₁₃₁₋₂₆₀ and characterization of interactions of this peptide with the SV40 origin has helped to define protein-DNA interactions at a eukaryotic origin (Joo, W. S., et al., 1997, J. Virol. 71:3972-3985). The affinity of the purified T-ag-obd for the SV40 origin was found to be comparable to that of full length T-ag.

The sequence of the SV40 core origin of replication is a 64 bp region of DNA (SEQ ID No.: 24) that can serve to initiate SV40 replication in vivo and in vitro (Deb, S., et al., 1986, Mol. Cell Biol. 6:1663-1670). The core origin contains three functional regions: (i) a central 27-bp region, termed site II, that contains four GAGGC pentanucleotides that serve as recognition sites for T-ag; (ii) an imperfect inverted repeat termed the early palindrome (EP); and (iii) an adenine/thymine rich (A/T) domain 13 (reviewed in Bullock, P. A., 1997, Crit. Rev. Biochem.Molec. Biol. 32:503-568). T-ag binds to duplex DNA containing 5′ GAGGC 3′ pentanucleotides (Tjian, R., 1978, Cell 13:165-179). Indeed, single 5′ GAGGC 3′ pentanucleotides support the formation of T-ag hexamers, and properly arranged pairs of pentanucleotides support double hexamer formation (and stable binding of the T-ag-obd₁₃₁₋₂₆₀).

Furthermore, stable binding of the T-ag-obd₁₃₁₋₂₆₀ to the SV40 core origin requires that pairs of the pentanucleotide recognition sites be separated by a single turn of the DNA double helix, and be positioned in a head-to-head orientation. The T-ag-obd₁₃₁₋₂₆₀ binds as a dimer to pentanucleotides 1 and 3. As full length T-ag oligomerizes on single stranded DNA, and T-ag-ob₁₃₁₋₂₆₀ interacts poorly with single stranded DNA (Joo, W. S., et al., 1997, J. Virol. 71:3972-3985), it is likely that regions of T-ag required to interact with single stranded DNA are located beyond the limits of the T-ag-obd. T-ag residues 121-135 are important for A/T untwisting (Chen, L., et al., 1997, J.Virol. 71:8743-8749), and failure to normally untwist the AIT tract correlates with a defect in origin unwinding.

Initiation of SV40 DNA replication is dependent on the assembly of two T-ag hexamers on the SV40 core origin (Joo, W. et al., 1998, Mol. Cell. Biol. 18:2677-2687). To further define the mechanism of oligomerization, the pentanucleotide requirements for T-ag assembly were investigated. Individual pentanucleotides support hexamer formation while particular pairs of pentanucleotides support the assembly of near wild type levels of T-ag double hexamers. T-ag double hexamers form on “active pairs” of pentanucleotides, and catalyze a set of structural distortions within the core origin. Additional footprinting experiments conducted on the four pentanucleotide-containing wild type SV40 core origin revealed that T-ag double hexamers, and the T-ag-obd, preferentially bind to pentanucleotides 1 and 3. Thus only two of the four pentanucleotides in the core origin are necessary for T-ag assembly and the induction of the structural changes in the core origin. Since all four pentanucleotides in the wild type origin are necessary for extensive DNA unwinding, the second pair of pentanucleotides must be utilized at a step subsequent to the initial assembly process.

Using band shift assays, the regions of the SV40 core origin required for stable assembly of T-ag and the T-ag-obd₁₃₁₋₂₆₀ were determined (Kim, H. Y., et al., 1999, J. Virol. 73:7543-7555). Binding of purified T-ag-obd₁₃₁₋₂₆₀ is mediated solely by interactions with site II. In contrast, T-ag binding and hexamer assembly requires a larger region of the core origin that includes both site II and an additional fragment of DNA that may be positioned on either side of site II. In the context of T-ag, the origin binding domain can engage the pentanucleotides in site II only if a second region of T-ag interacts with one of the flanking sequences. The requirements for T-ag double hexamer assembly are complex; the nucleotide co-factor present in the reaction modulates the sequence requirements for oligomerization. Only two pentanucleotides are required for T-ag or T-ag-obd binding, and only a subset of the SV40 core origin is required for assembly of T-ag double hexamers.

On a given modular unit, assembly is highly ordered (Sreekumar, K. R., et al., 2000, J. Virol. 74:8589-8600). For instance, on the penta 1, 3 and EP unit the first hexamer forms on pentanucleotide 1. Additional protein-protein interactions give rise to the second hexamer on pentanucleotide 3. On the penta 2, 4 and AT unit the first hexamer forms on penta 4 while the second forms on penta 2. Double hexamers can occupy only a single assembly unit at a given time. However, all four pentanucleotides are required for DNA unwinding and DNA replication. Therefore, the second set of pentanucleotides must be engaged during a post-assembly re-modeling step.

Regulation of SV40 DNA replication by phosphorylation of Thr124 includes the fact that the phosphorylation status of Thr124 does not have an impact on standard simple hexamer assembly on individual pentanucleotides. However, it is shown herein that Thr124 phosphorylation is necessary for double hexamer formation on individual assembly units. Mixing experiments demonstrate that the presence of the protein with mutation T124A inhibits T-ag assembly. Thus the T124A mutant is a dominant negative inhibitor of DNA unwinding and DNA replication (Weisshart, K., et al., 1999, J. Virol. 73:2201-2211). Further, cell cycle dependent phosphorylation of T-ag on Thr124 controls the assembly of the second hexamer. In summary, using “single assembly units” as substrates, direct evidence is provided that formation of T-ag double hexamers is controlled by phosphorylation of Thr124.

In the examples that follow, peptides were designed and synthesized to contain NLS regions from a variety of viral and human proteins, in particular, T-ag of SV40, and each of the proteins encoded by the human tumor suppressor p53 gene (SEQ ID NO: 1), the E1 protein encoded by bovine papillomavirus (SEQ ID NO: 2), and a set of control peptides. Binding assays of these peptides were performed with a 64 bp oligonucleotide derived from the SV40 core origin (SEQ ID NO: 24). Examples herein show that as with the peptide derived from the T-ag NLS region, the peptides derived from the p53 and E1 NLS regions bind to double-stranded DNA. The 25 amino acid long peptide derived from the E1 protein, SEQ ID NO: 2, binds to DNA very strongly, as indicated by the fact that the experimental observation of binding herein does not require cross-linking with glutaraldehyde. A larger version of the T-ag NLS peptide containing 17 amino acids also was found to remain bound to DNA in the absence of glutaraldehyde. Single stranded DNA was bound poorly by p53 and E1 NLS region peptides.

As the 25 amino acid long NLS peptide derived from papillomavirus E1 (SEQ ID NO: 2) was found to be a very good DNA binder, therefore the ability of this peptide to block DNA replication in an in vitro SV40 replication assay was tested. Addition of 20 nM of this peptide to a reaction was found to completely block replication. Additional studies indicated that the papillomavirus based NLS is not only the best DNA binder, but also the best inhibitor of DNA replication of peptides tested herein.

Further, nitrocellulose filter binding techniques were used to analyze the interaction between the NLS-peptides and DNA. These experiments confirmed and extended previous gel based observations regarding the ability of these peptides to bind to DNA. Filter binding assays are very fast and accurate; therefore, this technique was used to analyze the interaction of non-T-ag based NLS peptides with DNA, e.g., the E1 NLS peptide (SEQ ID NO: 2) described above.

As these peptides contain the NLS region, peptide variants and peptide mimetics can be developed that can similarly be targeted to the nucleus. Variants and mimetics can be synthesized and immobilized on arrays, and tested for binding to DNA targets which can be detected by conventional labeling of DNA with radioactive or fluorescent adducts or tags (see for example, U.S. Pat. No. 5,652,099). Samples of peptides can be immobilized at addressible locations or spots on a solid substrate, for example a glass slide, by chemical methods in which peptide derivatives are synthesized that react with an underivatized glass slide (for example, as shown in PCT/US01/15446, WO 02/092615). Peptide arrays and detection of ligands bound to spots within the array are described in U.S. Pat. Nos. 6,545,758 and 6,567,163.

In an alternative method, phage display techniques can be used to select peptide derivatives that bind site specifically to duplex DNA. Yet another method is selecting peptides that bind to particular target sequences using a modified two-hybrid system (Vojtek et al., 1993, Cell 74: 214). For an initial set of experiments, the DNA binding domain of the “bait” is replaced with the E1 NLS peptide. The E1 NLS peptide will bind to DNA and generate blue colonies in the presence of the “prey”. Then libraries of NLS based peptides are used to identify peptides that bind to particular target sequences.

It is thus shown here that peptides derived from NLS regions of proteins can bind to DNA. Further, if peptides can be targeted to particular sites on DNA, peptide mimetics derived from these compounds may be developed as an important class of drugs. These peptides may serve many other useful purposes, for example, use as probes for diagnostic purposes and for research.

Methods and Uses

The therapeutic compounds of the invention can be used to treat a viral infection or a cancer, by down-regulating (modulating) expression of a gene associated with that condition. Therapeutic compounds of the invention, while characterized as binding SV40 double-stranded DNA, can have increased affinity for nucleotide sequences of DNA in genes associated with other viral or tumor-related diseases.

A pharmaceutically acceptable carrier includes any and all solvents, dispersion media, coatings, antimicrobials such as antibacterial and antifungal agents, isotonic and absorption delaying agents and the like that are physiologically compatible. Preferably, the carrier is suitable for intravenous, intramuscular, oral, intraperitoneal, transdermal, or subcutaneous administration. The active compound can be coated in a material to protect it from inactivation by the action of acids or other adverse natural conditions.

A composition of the present invention can be administered in a variety of formulations and by a variety of methods known in the art as will be appreciated by the skilled artisan. A peptide can be supplied as a salt, such as an acetate form, that is reconstituted in aqueous solution and administered to a patient subcutaneously. The peptide, and an additional active compound, or peptide variant or mimetic as described herein, to be administered in combination with or as an alternative embodiment to the peptide, can further be prepared with carriers that will protect it against rapid release, such as a controlled release formulation, including implants, transdermal patches, and micro-encapsulated delivery systems. Many methods for the preparation of such formulations are patented and are generally known to those skilled in the art. See, e.g., Sustained and Controlled Release Drug Delivery Systems, J. R. Robinson, Ed. Marcel Dekker, Inc., NY (1978).

Therapeutic compositions for delivery in a pharmaceutically acceptable carrier are sterile, and are preferably stable under the conditions of manufacture and storage. The composition can be formulated as a solution, microemulsion, liposome, or other ordered structure suitable to high drug concentration. Dosage regimens can be adjusted to provide the optimum desired response (e.g., a therapeutic response). For example, a single bolus can be administered, several divided doses can be administered over time, or the dose can be proportionally reduced or increased as indicated by the exigencies of the disease situation.

In various embodiments of the methods provided herein, a suitable daily dose of a therapeutic synthetic peptide composition is administered that will be the lowest effective dose to produce a therapeutic effect, for example, mitigation of symptoms. The therapeutic peptide compounds of the invention are preferably administered at a dose per subject per day of at least 2 mg, at least 5 mg, at least 10 mg or at least 20 mg as appropriate minimal starting dosages. In general, the compound of the effective dose of the composition of the invention can be administered in the range of 50 to 400 micrograms of the compound per kilogram of the subject per day.

A physician or veterinarian having ordinary skill in the art can readily determine and prescribe the effective dose of the pharmaceutical composition required. For example, the physician or veterinarian could start doses of the compound of the invention employed in the pharmaceutical composition at a level lower than that required in order to achieve the desired therapeutic effect, and increase the dosage with time until the desired effect is achieved.

A desired therapeutic effect can be observed as an increased period of remission from cancer or a viral infection, for example, due to a decreased expression of the gene over a period of time. Another desired therapeutic effect can be remission in symptoms such as tumor growth, viral titer, antigenic titer, pain, dizziness, fatigue, visual and cognitive disturbances as noted herein. Remissions of symptoms can be self-reported by the patient, or can be quantitatively detected by standard measurements of biochemical markers or nucleic acid levels, known to practitioners in the art of treating cancer or viral infections.

In other embodiments the pharmaceutical composition includes also at least one additional therapeutic agent. Thus in a method of the invention, the pharmaceutical composition can be administered as part of a combination therapy, i.e. in combination with an additional agent or agents. Examples of materials that can be used in combination with peptides herein for treatment of cancer or a viral infection as additional therapeutic agents include: an antibody or an antibody fragment conjugate that can bind specifically to a transformed or infected cell; an enzyme inhibitor which can be a protein, such as α₁-antitrypsin or aprotinin; an enzyme inhibitor which can be a cyclooxygenase inhibitor; an engineered binding peptide protein, for example, an engineered protein that is a protease inhibitor such an engineered inhibitor of kallikrein; an antibacterial agent which can be an antibiotic such as amoxicillin, rifampicin, erythromycin; an antiviral agent which can be a low molecular weight chemical such as acyclovir; a steroid, for example a corticosteroid, or a sex steroid such as progesterone; a non-steroidal anti-inflammatory agent such as aspirin, ibuprofen, or acetaminophen; an anti-cancer agent such as methotrexate or adriamycin; or a cytokine.

The additional therapeutic agent can be a cytokine, a term which as used herein includes without limitation agents which are naturally occurring proteins or variants and which function as growth factors, lymphokines, interferons such as β-interferon, tumor necrosis factors, angiogenic or antiangiogenic factors, erythropoietins, thrombopoietins, interleukins, maturation factors, chemotactic proteins, or the like. Preferred combination therapeutic agents to be used with the composition of the invention include additional anti-viral or anti-cancer agents as indicated by the condition to be treated. A therapeutic agent to be used with the composition of the invention can be an engineered binding peptide or protein, known to one of skill in the art of remodeling a protein that was isolated by virtue of having been covalently attached to a virion coat protein as a genetic fusion (Ladner, R. et al., U.S. Pat. No. 5,233,409; Ladner, R. et al., U.S. Pat. No. 5,403,484), and can be made according to methods known in the art. A protein such as an engineered protein that binds any of a variety of other targets can be engineered and used in the present invention as a therapeutic agent in combination with a peptide of the invention.

An improvement in the symptoms as a result of administration of any of the compositions herein is noted by a reduction in symptoms such as the symptoms of a viral infection or a cancer by at least about 20%, by at least about 40%, by at least about 60%, or by at least about 80%, relative to untreated subjects. Cure or complete remission, or improvement of symptoms, can be noted by increased life span, elimination of relapsing episodes, and significantly improved overall health of the patient.

The invention having been full described, the following Examples are used to illustrate certain embodiments that are not intended to be further limiting. The contents of all citations are hereby incorporated herein by reference in their entirety.

### EXAMPLES

The following materials and methods were used throughout the Examples herein.

Commercial supplies of enzymes, DNA, reagents, oligonucleotides and peptides. T4 polynucleotide kinase was purchased from Gibco-BRL. Plasmid pBR322 DNA, used as competitor DNA, was purified according to standard procedures (Sambrook, J., et al., 1989, Cold Spring Harbor Laboratory, Cold Spring Harbor, N.Y.) and digested with Hae III purchased from New England Biolabs.

Oligonucleotides were synthesized on an Applied Biosystems 394 DNA synthesizer, purified by electrophoresis through 10% urea-polyacrylamide gels and isolated as described (Sambrook, J., et al., 1989, Cold Spring Harbor Laboratory, Cold Spring Harbor, N.Y.; Sreekumar, K. A., et al., 2000, In, L. Raptis (ed.), Methods in Molecular Biology: SV40 Protocols, Humana Press, Totowa, N.J. p.49-67). Double stranded oligonucleotides, labeled with ³²P at the 5′ termin, were prepared using standard procedures.

Purification of T-ag. SV40 T-ag was produced using a baculovirus expression vector containing the T-ag-encoding SV40 A gene (O'Reilly, D. R., et al., 1988, J. Virol. 62:31 09-3119) and purified using immunoaffinity techniques (Dixon, R. A. F., et al., 1985, J. Virol. 53:1001-1004, Simanis, V., et al., 1985, Virol. 144:88-100). Purified T-ag was dialyzed against T-ag storage buffer (20 mM Tris-HCl pH 8.0, 50 mM NaCl, 1 mM EDTA, 1 mM dithiothreitol (DTT), 0.1 mM phenylmethylsulfonyl fluoride, 0.2 μg of leupeptin per ml, 0.2 μg of antipain per ml and 10% glycerol) and frozen at −70° C. until use.

Synthesis and purification of NLS based peptides. Peptides were synthesized at the Tufts Core Facility on an Applied Biosystems 431A Peptide Synthesizer using solid phase methodologies. After cleavage and deprotection, the samples were ether precipitated, re-suspended in dH2O, lyophilyzed and purified by reverse phase HPLC. The peptides were re-suspended to 10 mM in equal volumes of 0.5% NH₄HCO₃ and 233 T-ag storage buffer. Re-suspended peptides were frozen at −20° C. until use.

Electrophoretic mobility shift assays (EMSA). Double stranded oligonucleotides, ³²P labeled at 5′ termini, were prepared using standard procedures (Sambrook, J., et al., 1989, Cold Spring Harbor Laboratory, Cold Spring Harbor, N.Y.; Sreekumar, K. A., et al., 2000, p. 49-67; In, L. Raptis (ed.), Methods in Molecular Biology; SV40 Protocols; Humana Press; Totowa, N.J.). EMSA rections were conducted using conditions for SV40 in vitro replication (Wobbe, C. R., et al., 1985, Proc. Natl. Acad. Sci. USA 82:5710-571 4). The reactions (20 μl) contained 7 mM MgCl₂, 0.5 mM DTT, 4 mM AMP-PNP, 40 mM creatine phosphate (pH 7.6), 0.48 μg of creatine phosphate kinase, 5 μg of bovine serum albumin, 0.8 μg of Hae III-digested pBR322 (about 6 pmol; used as a nonspecific competitor), about 25 fmol of double stranded oligonucleotide, T-ag and the indicated amounts of peptide. After a 20-min incubation at 37° C., glutaraldehyde (0.1 % final concentration) was added and the reaction products were further incubated for 5 min. The reactions were stopped by the addition of 5 μl of 6× loading dye II (15% Ficoll, 0.25% bromophenol blue, and 0.25% xylene cyanol; Sambrook, J., et al., 1989, Cold Spring Harbor Laboratory, Cold Spring Harbor, N.Y.) to the reaction mixtures. Samples containing T-ag were applied to 3.5 to 12% gradient polyacrylamide gels and electrophoresed in 0.5% Tris-borate-EDTA (TBE) pH 8.4 for about 1.5 h (10 watts).

Peptide based band shift reactions were identical to those described above except that T-ag was omitted from the reactions and the final glutaraldehyde concentration was reduced to 0.05 %. Moreover, the samples were loaded on 8% polyacryamide gels and electrophoresed in 0.5% TBE pH 8.4 for about 3h (about 200 volts). The gels were dried on Whatman 3 MM paper, subjected to autoradiography and quantitated using a Molecular Dynamics PhosphorImager (Sunnyvale, Calif.).

Nitrocellulose filter binding reactions. Assay for the ability of NLS based peptides to bind to DNA were performed using published methods (Sreekumar, K. A., et al., 2000, In, L. Raptis (ed.), Methods in Molecular Biology: SV40 Protocols; Humana Press, Totowa, N.J. p. 49-67). Reactions (20 μl) contained 7 mM MgCl₂, 0.5 mM DTT, 4 mM AMP-PNP, 40 mM creatine phosphate (di-Tris salt pH7.7), 0.48 μg of creatine phosphokinase, 0.2 mg of bovine serum albumin per ml, 0.8 μg of Hae III-digested pBR322 DNA, about 25 fmol of a given oligonucleotide (about 10⁶ cpm/pmol) and the indicated peptide. After incubation for 20 min at 37° C., the mixtures were filtered under suction through alkali-treated nitrocellulose filters (Millipore type HAWP; pore size, 0.4 μm; stored in 100 mM Tris-HCl, pH7.5). The filters were then washed with 5 ml of 100 mM Tris-HCl, pH 7.5, and were dried and counted in a Beckman LS 3801 scintillation counter.

DNA replication reactions. SV40 in vitro replication reactions were conducted as described by Wobbe et al 1985, Proc. Natl. Acad. Sci. USA 82:5710-5714 (see also, Bullock, P. A., et al., 1998, p. 223-243. In S. Cotterill (ed.), Eukaryotic DNA Replication; A practical Approach, Oxford University Press, Oxford). Reaction mixtures (30 μl) contained 7 mM MgCl₂, 0.5 mM DTT, 4 mM ATP, 40 mM creatine phosphate (di-Tris salt pH7.7), 1.4 μg of creatine phosphokinase, 12.5 μg/ml pSV01ΔEP, dATP, dGTP, and dTTP (100 μM each), CTP, GTP, and UTP (200 μM each), [α-³²p] dCTP (20 μM, about 5 cpm/fmol) and the indicated peptides.

### Example 1

### Role of an NLS Based Peptide in Inhibiting T-ag Oligomerization Events

The T-ag genes of 7 different mammalian papovaviruses, SV40, BKV, JCV, LPV, HaPV, PyV, and KV (see Table 1), contain a consensus of a Cdk kinase recognition site followed by one or two proline residues, and an NLS comprising a highly basic region of two to four arg or lys residues (Pipas, J. 1992 J. Virol. 66:3979-3985) characteristic of the NLS consensus (SEQ ID NOs: 3-10). From the examples herein, this consensus of the Cdk/NLS site is shown to form the core of a peptide that binds to DNA, and inhibits double hexamer assembly of T-ag protein and inhibits helicase activity and concomitant ATPase of T-ag.

The human p53 protein contains several NLS sequences, one of which is linked to a Cdk motif. Phosphorylation of p53 Ser315 by cyclinB/Cdc2 influences the selectivity of p53 binding (Steegenga et al. J. Mol. Biol. 263:103-113), increasing the binding of p53 to p21Waf1 gene, without affecting binding to any of the ribosomal gene cluster, the SV40 replication control region or the muscle creatine kinase enhanced. A peptide from this region of p53 was synthesized (SEQ ID NO: 1) and tested to determine if it binds to DNA. This peptide binds to double-stranded, but not single-stranded DNA.

The T-ag derived peptides used herein are shown in Table 2A; peptides include the sets of peptides of length 9 mer, 13 mer and 17 mer. Each set contains peptides that include the amino acid sequences of peptides 1 and 2 (SEQ ID NOs: 25 and 26, respectively), and control peptides 3 and 4 (SEQ ID NOs: 27 and 28, respectively). Peptide 1 differs from peptide 2 in that Thr124 is phosphorylated on peptide 1 but not on peptide 2. In control peptides, the amino acids sequences positioned on each side of Thr124 in peptides 1 and 2 were switched. In Table 2, the amino acid residues derived from the SV40 NLS are shown in bold; those forming the consensus Cyclin-Cdk recognition site are underlined.

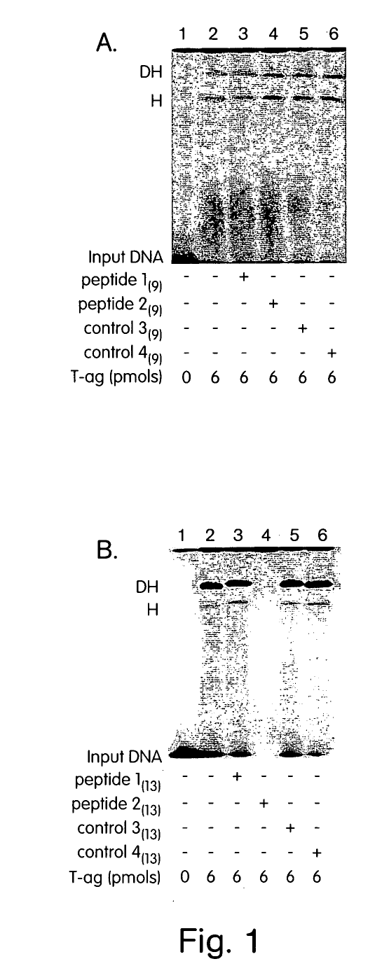

Interference by the 9-mer set of peptides (Table 2) with formation of hexamers and double hexamers on an oligonucleotide containing the SV40 core origin was initially analyzed (FIG. 1 Panel A). Binding of T-ag to this oligonucleotide resulted in the formation of hexamers and double hexamers (lane 2). Lanes 3-5 reveal that addition of any of the 9-mer set of peptides (20 nM of each; final concentration of 1 mM) did not affect the assembly of T-ag.

Band shift reactions conducted in the presence of each of the 13-mer set of peptides, at final concentrations of 1 mM, are shown in FIG. 1 Panel B (lanes 3-6). Lanes 3, 5 and 6 show that each of peptide 1₁₃ (SEQ ID NO: 12), peptide 3₁₃ (SEQ ID NO: 13), and peptide 4₁₃ (SEQ ID NO: 14) had little or no effect on T-ag oligomerization. However, the presence of peptide 2₁₃ (SEQ ID NO: 3), inhibited both hexamer and double hexamer formation. It is noted that while the 9-mer and 13-mer sets of peptides contain the complete cyclin/cyclin-dependent kinase consensus sequence ([S/T]PX[R/K]; Songyang, Z. et al. 1994 Curr. Bio. 4:973-982), the 13-mer peptides each contain two additional residues of the T-ag NLS (Table 2A).

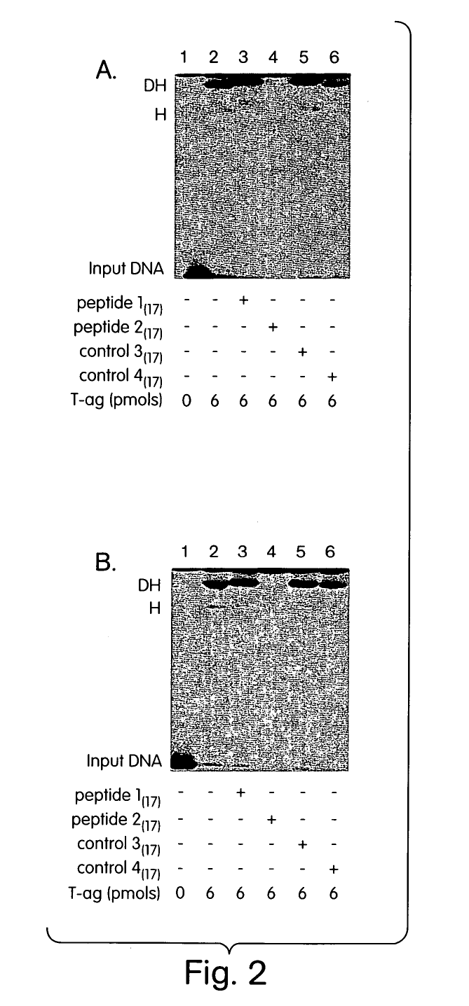

Analysis of data obtained with the 17 mer set of peptides (FIG. 2 Panel A, lanes 3-6) shows that as with the 13-mer set of peptides, there was no inhibition by peptides 1₁₇ (SEQ ID NO: 15) or peptide 3₁₇ (SEQ ID NO: 16) when reactions were conducted at a final concentration of 1 mM. However, T-ag assembly was completely blocked by peptide 2₁₇ (SEQ ID NO: 11) and to a lesser extent by peptide 4₁₇ (SEQ ID NO: 17). To determine whether inhibition by peptide 4₁₇ might be affected by a relatively high peptide concentration used in that analysis, an experiment as in FIG. 2 Panel A was performed using a lower final concentration of peptide (0.5 mM FIG. 2 Panel B, lanes 3-6). This Figure shows that peptide 2₁₃ (SEQ ID NO: 3) inhibited T-ag assembly events (lane 4), and that peptide 4₁₇ (SEQ ID NO: 17) did not inhibit, at the lower concentration of peptide (lane 6).

It is here shown that peptides derived from the NLS region of T-ag can block T-ag assembly events, provided they do not contain a phosphate at Thr124. Moreover, as the control peptides having rearranged sequences did not inhibit the assembly events, these data further demonstrate that inhibition is dependent on the correct order of the NLS element.

In several additional analyses, it was observed that the mobility of the input DNA had become heterogeneous, i.e., the DNA was heterogeneous in size in those lanes that contained peptide 2₁₃ (SEQ ID NO: 3) or peptide 2₁₇ (SEQ ID NO: 11). These observations raised the possibility that peptide 2₁₃ and peptide 2₁₇ blocked T-ag assembly events because of their ability to bind DNA.

### Example 2

### Determining Whether T-ag derived NLS Peptides Bind to DNA

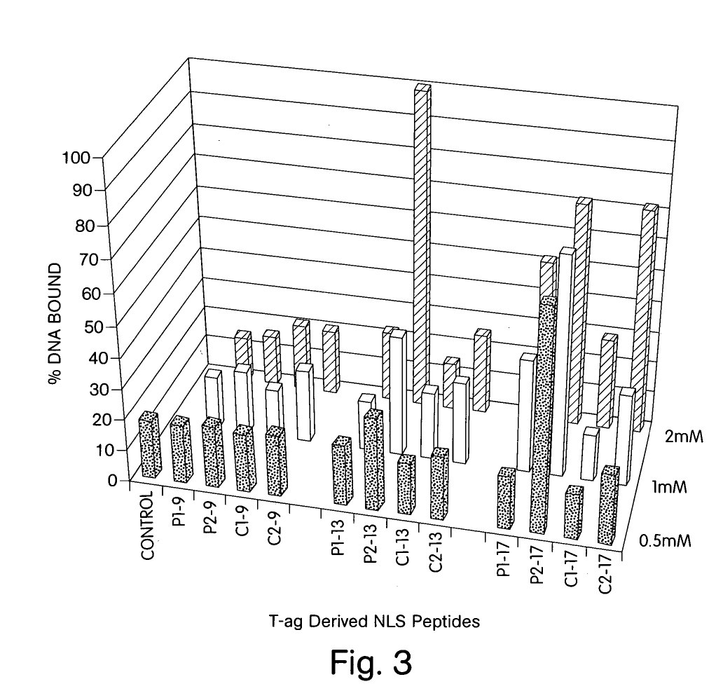

To determine whether certain of the peptides herein bind to DNA, filter binding assays with peptides shown in Table 1A were conducted. Results from these assays (FIG. 3) show that, over a range of peptide concentrations (0.5 mM to 2 mM), the 9-mer peptides did not bind at levels above background to an oligonucleotide containing the SV40 core origin. However, with the 13-mer set of peptides, it was found that peptide 2₁₃ (SEQ ID NO: 3) binds to DNA. At a final concentration of 2 mM, saturation of binding was observed for peptide 2₁₃. Other peptides such as peptide 1₁₃ (SEQ ID NO: 12) did not bind DNA at significant levels. Low levels of binding were observed with peptide 4₁₃ (SEQ ID NO: 14) at high concentrations of peptide (e.g., 1 mM and 2 mM). DNA binding was also detected with the 17-mer set of peptides. At the lower concentration of 0.5 mM, significant binding was observed only with peptide 2₁₇ (SEQ ID NO: 11). Peptide 2₁₃ bound relatively poorly at 0.5 mM, while peptide 2₁₇ bound significant levels of DNA at this concentration.

These observations indicate that peptide 2₁₇ (SEQ ID NO: 11), which contains the complete SV40 NLS, has a relatively high affinity for binding to DNA. Further, lack additional binding as the concentration of peptide 2₁₇ was increased indicates that the reactions at 1 mM and 2 mM were saturated. Consistent with this finding, at higher peptide concentrations additional members of the 17-mer set of peptides (e.g., peptide 1₁₇; SEQ ID NO: 15) and peptide 4₁₇; SEQ ID NO: 17) were observed to bind to DNA. As these reactions were conducted using “band shift” conditions (see Examples), the reactions contained significant amounts of pBR322 as a non-specific competitor. Thus, the data from these assays can establish relative binding affinities rather than to quantitate the affinities.

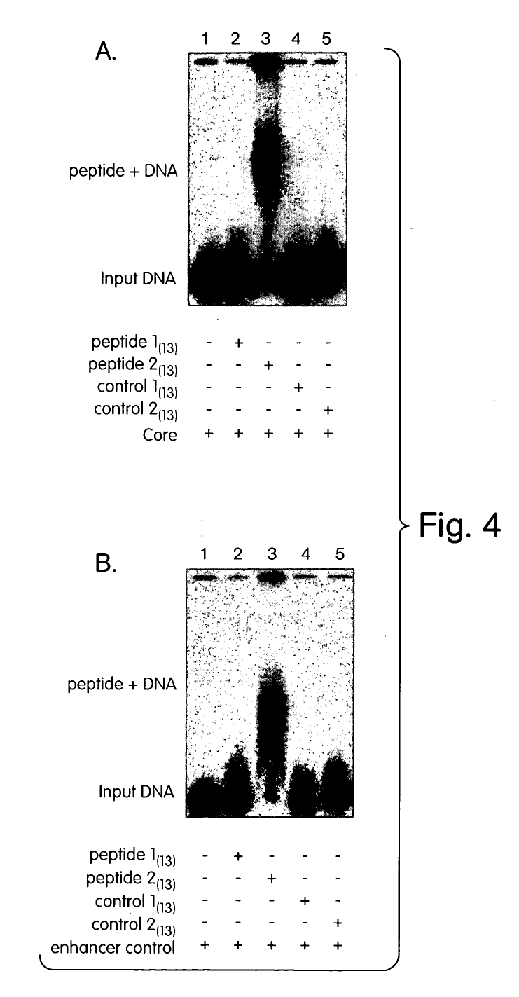

To further characterize the peptide/DNA interactions, additional peptide band shift experiments were conducted (FIG. 4 Panel A). The reactions were performed in the presence of a duplex oligonucleotide containing the core origin; the mobility of the 64 bp core origin (SEQ ID NO: 24) in the absence of peptide is shown in lane 1. The reaction products formed when the 13-mer set of peptides was included in the reactions are shown in lanes 2-5. Peptide 2₁₃ (SEQ ID NO: 3) among this set was found to bind to DNA (lane 3).

To determine whether a high degree of sequence specificity is required for peptide binding, analyses were performed using a control oligonucleotide (the 64 bp enhancer control, SEQ ID NO: 29). Peptide 2₁₃ (SEQ ID NO: 3) bound to the 64 bp enhancer control (FIG. 4 Panel B; lanes 2-5). Binding of peptide 2₁₃ is therefore not highly sequence specific. As found with the reactions conducted in the presence of the core origin (FIG. 4 Panel A), the other members of the 13-mer set of peptides did not bind to the 64 bp enhancer control under these conditions (FIG. 4 Panel B). Lanes containing peptide 2₁₃ retained considerable of material in the sample input wells (e.g., lane 3) indicating that in addition to peptide binding to these DNA substrates, aggregates were also formed.

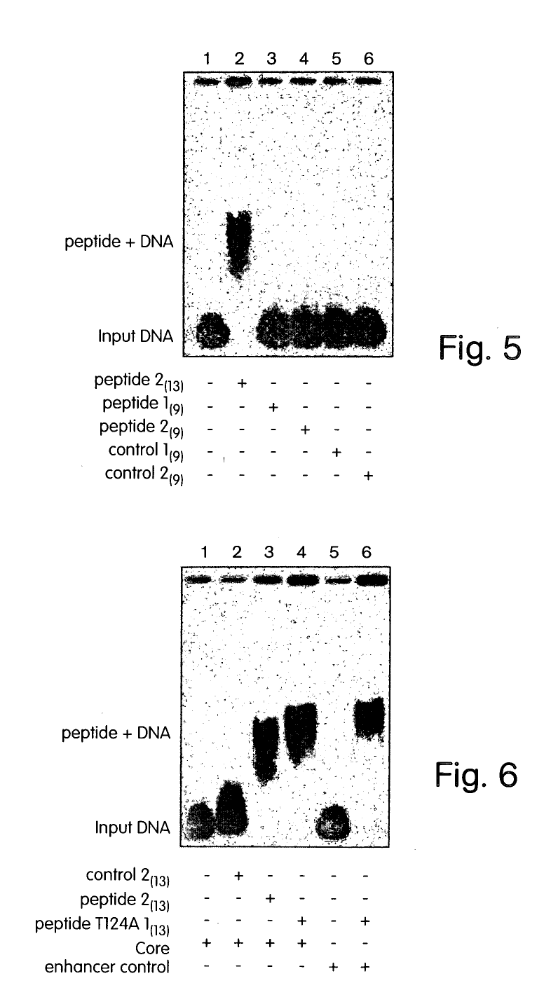

Analyses were performed with each of the 9-mer set of peptides (FIG. 5) at a peptide concentration that is twice that used to detect interactions of the 13-mer set of peptides with DNA (2 mM). As a positive control, a reaction was conducted with peptide 2₁₃ (lane 2); the location of the peptide/DNA complex described in FIG. 4 is indicated. Lanes 3-6 of FIG. 5 show the reactions conducted with the 9-mer set of peptides (SEQ ID NOs: 25-28). In contrast to the 13-mer set of peptides, binding was not detected with peptide 2₉ (SEQ ID NO: 26) or other members of the 9-mer set.

Data herein demonstrate that an approximate lower limit of length is required for peptide binding, which is about 13 residues. The data also indicate that the ability of peptide 2₁₃ (SEQ ID NO: 3) to inhibit T-ag oligomerization (as shown in FIGS. 1-2) is due to its ability to bind to DNA.

### Example 3

### Determining if Thr124 is Necessary for the Interaction Between Peptide 2₁₃ and DNA

The role of Thr124 in the regulation of the process of peptide binding to DNA was examined to determine if this threonine residue is necessary for DNA binding. A derivative of peptide 2₁₃ having a replacement of Thr124 by an alanine was synthesized and is referred to herein as peptide T124A₁₃; SEQ ID NO: 20).

Data obtained from a set of reactions performed with this molecule are presented in FIG. 6. The reactions in lanes 1-4 were conducted with a duplex oligonucleotide containing the SV40 core origin (SEQ ID NO: 24). The reaction shown in lane 1 was conducted in the absence of peptide, while the reactions displayed in lanes 2 and 3 were conducted in the presence of peptides serving as negative and positive controls, peptide 4₁₃ (SEQ ID NO: 14) and peptide 2₁₃ (SEQ ID NO: 3), respectively. Consistent with data above, peptide 2₁₃ was found to bind to DNA, while peptide 4₁₃ did not. The reaction in lane 4 was conducted with peptide T124A₁₃ (SEQ ID NO: 20). As with peptide 2₁₃, it is apparent that peptide TI24A₁₃ bound to DNA. The reactions in lanes 5 and 6 were conducted with the 64 bp enhancer control oligonucleotide (SEQ ID NO: 29). The reaction in lane 6 establishes that as with peptide 2₁₃, the binding of peptide T124A₁₃to DNA does not require a large degree of sequence specificity.

Residue Thr124 is shown by the date herein as not necessary for DNA binding per se, and is mainly involved in the regulation of this process as is shown below.

### Example 4

### An NLS Peptide from Papillomavirus Initiator is Able to Bind to DNA

To establish whether NLS peptides from a gene other than SV40 T-ag would bind to DNA, a 25 residue long peptide from the NLS for Bovine Papillomavirus E1 (SEQ ID NO: 2; Lentz, M. R., et al., 1993, J. Virol. 67:1414-1423) was synthesized. A control peptide was designed by inverting the amino acid sequences of residues on either side of Gly 96 (Table 2) and was synthesized (SEQ ID NO: 19).

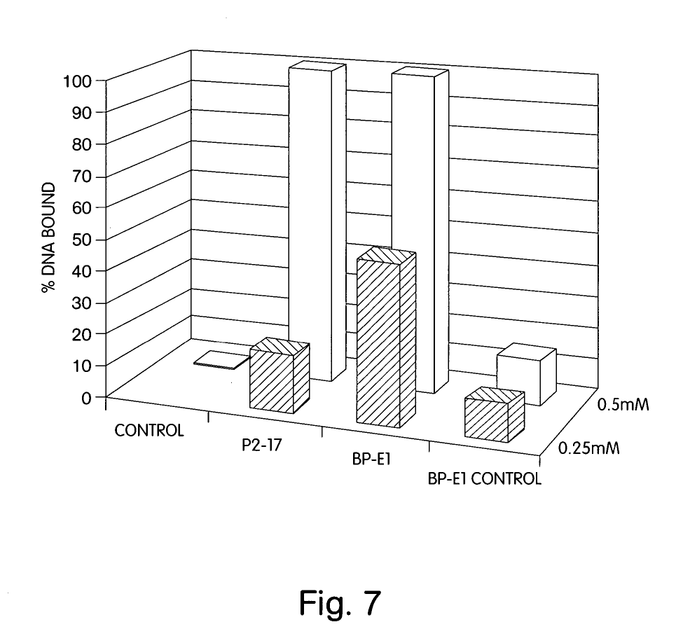

To establish if the BP-E1 based NLS peptide bound to DNA, filter binding experiments were conducted. FIG. 7 shows that the E1 based NLS peptide bound to DNA. At low peptide concentrations (0.25 mM), the BP-E1 peptide was found to be a better DNA binder than peptide 2₁₇ (SEQ ID NO: 11) from T-ag. Further, binding was so strong that EMSA procedures could be performed with the BP-E1 peptide, in the absence of cross-linking with glutaraldehyde. The control E1 peptide having inverted sequences bound to DNA at much reduced levels.

It is here shown that peptides derived from the NLS regions of each of Bovine Papillomavirus E1 and SV-40 T-ag bind to DNA in a manner that exhibits some specificity of the amino acid sequence. Thus binding of peptides to DNA is not due merely to the charge of the peptides, since the control peptides having the same residues in different order and thus the same charge but a rearranged sequence do not bind. Another control peptide, SEQ ID NO: 30 having amino acid sequence KKRRKRVKLVGPSTSEQSNASESSG, retains ability to inhibit replication of DNA.

### Example 5

### NLS Based Peptides Block DNA Replication

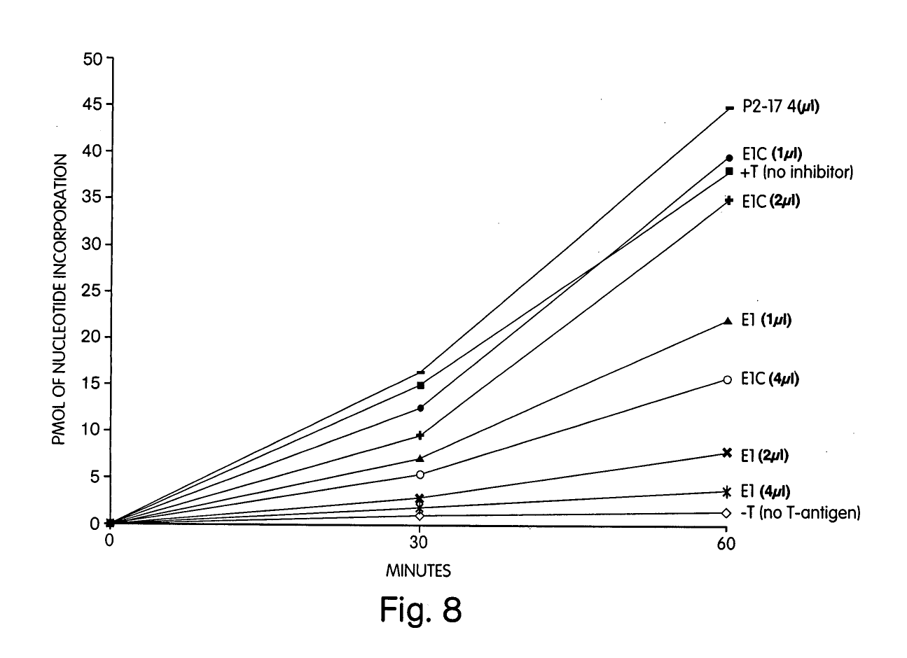

SV40 in vitro replication assays were used to determine whether peptides with higher affinities for DNA binding (e.g. T-ag peptide 2₁₇ (SEQ ID NO: 11) and BP-E1₂₅ (SEQ ID NO: 2) can inhibit replication (FIG. 8). Control reactions were conducted in the absence and presence of T-ag.

In the presence of T-ag, approximately 40 pmol of DNA synthesis was observed. Presence of the BP-E1₂₅ peptide at all concentrations tested inhibited this SV40 replication. Addition of the BP-E1₂₅ peptide at a concentration of about 600 μM completely blocked viral DNA replication.

The control BP-E1₂₅ peptide was not an efficient inhibitor of replication, indicating that inhibition is not a simple function of the peptide charge. In contrast to the BP-E1₂₅ peptide, peptide 2₁₇ from T-ag was not an efficient inhibitor of DNA replication. That BP-E1₂₅ peptide inhibits replication, but peptide 2₁₇ does not, may be related to the relatively high affinity of the BP-E1₂₅ peptide for DNA.

An NLS based peptide from Bovine papillomavirus was thus found to inhibit in vitro DNA replication reactions here conducted using HeLa human cell crude extracts. As a significant extent of non-specific DNA binding is found (e.g., FIG. 4 Panel B), it is likely that the inhibition is due, at least in part, to non-specific binding to DNA. Finally, a control peptide having amino acid sequence KKRRKRVKLVGPSTSEQSNASESSG (SEQ ID NO:30), was found herein to be a good inhibitor of DNA replication.

The examples presented herein demonstrate that peptides derived from the NLS region of SV40 T-ag are DNA binding elements. The SV40 NLS region is nearly identical to the NLS regions of viruses BK and JC (Pipas, J. M., 1992, J. Virol. 66:3979-3985), thus it is likely that NLS based peptides derived from these human pathogens would also function as DNA binding elements. As it is shown further herein that the NLS based peptides lose their ability to bind DNA when Thr124 is phosphorylated, phosphorylation of Thr124 acts as a switch that regulates DNA binding.

The ability to bind to DNA is not limited to peptides derived from the T-ag NLS region, as a peptide containing the bipartite NLS from Bovine papillomavirus E1 is also a DNA binding element. Furthermore, the BP-E1₂₅ peptide inhibits in vitro DNA replication reactions. This is a surprising result given that Hela cell crude extracts contain diverse populations of nucleic acids that are likely to compete for peptide binding. Furthermore, it was anticipated that the BP-E1₂₅ peptide would not function as an inhibitor owing to degradation or inactivation by phosphorylation. Why T-ag derived peptide 2₁₇ is unable to inhibit replication has yet to be established. However, without being bound by any particular mechanism, one explanation is that it does not have as great a binding affinity as the BP-E1₂₅ peptide. BP-E1₂₅ as shown herein binds to DNA at a lower concentration than does peptide 2₁₇. Alternatively, the BP-E1₂₅ peptide may adopt a conformation that stabilizes it in HeLa cell crude extracts.

Peptides containing Zn finger motifs have also been reported to bind to DNA with limited sequence specificity (Kim, J. S., et al., 1998, Proc. Natl. Acad. Sci. USA 95:2812-2817). Further, a 35 residue alpha helix containing peptide has high affinity for a target ATGAC sequence (Chin, J. W., et al., 2001, J. Am. Chem. Soc. 123:2929-2930 Science, 2001, 291, 204). The peptides herein are smaller (13-25 residues), and are derived from the nuclear localization signal (NLS) of the protein encoded by each respective gene. Thus, the NLS peptides described herein have an address or a “Zip code” that targets them to the nucleus. In contrast to the lengths in number of amino acids of previously described peptides capable of binding to DNA, the NLS based peptides herein are the smallest DNA species, and are able to localize to a eukaryotic nucleus, and are the only peptides with capability of binding to DNA that can be regulated by phosphorylation among these DNA species.

The SV40 T-ag NLS region contains a CcN motif, a motif known to be conserved in many nuclear proteins (Jans, D. A., et al., 1998, Medicinal Res. Rev. 18:189-223), the motif consisting of a casein kinase 2 site, a cyclin-dependent kinase 2 site and a monopartite NLS. All three components of the CcN motif can have roles in phosphorylation dependent regulation of T-ag nuclear translocation (Jans, D., et al. 1991. J. Cell Biol. 115:1203-1212; Jans, D. A., et al., 1994, Oncogene 9:2961-2968; Rihs, H. P., et al., 1991, EMBO J. 10:633-639). Nuclear translocation of several other proteins is regulated by phosphorylation of CcN motifs (e.g., Lam, M., et al., 1999, J. Biol. Chem. 274:18559-18566; Zhang, F., et al., 2000, Proc. Natl. Acad. Science 97:12577-12582). Phosphorylation of these sites is shown herein to control ability to bind DNA.

### Example 6

### Requirement for a Non-Phosphorylated Peptide for Inhibition of T-ag Assembly

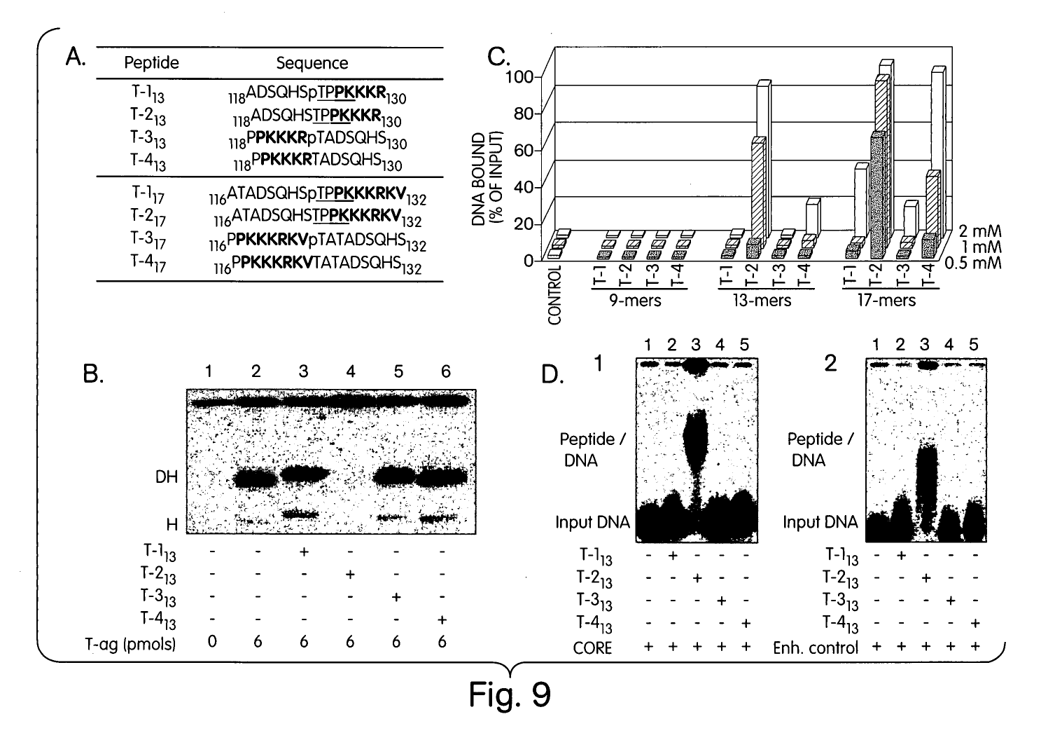

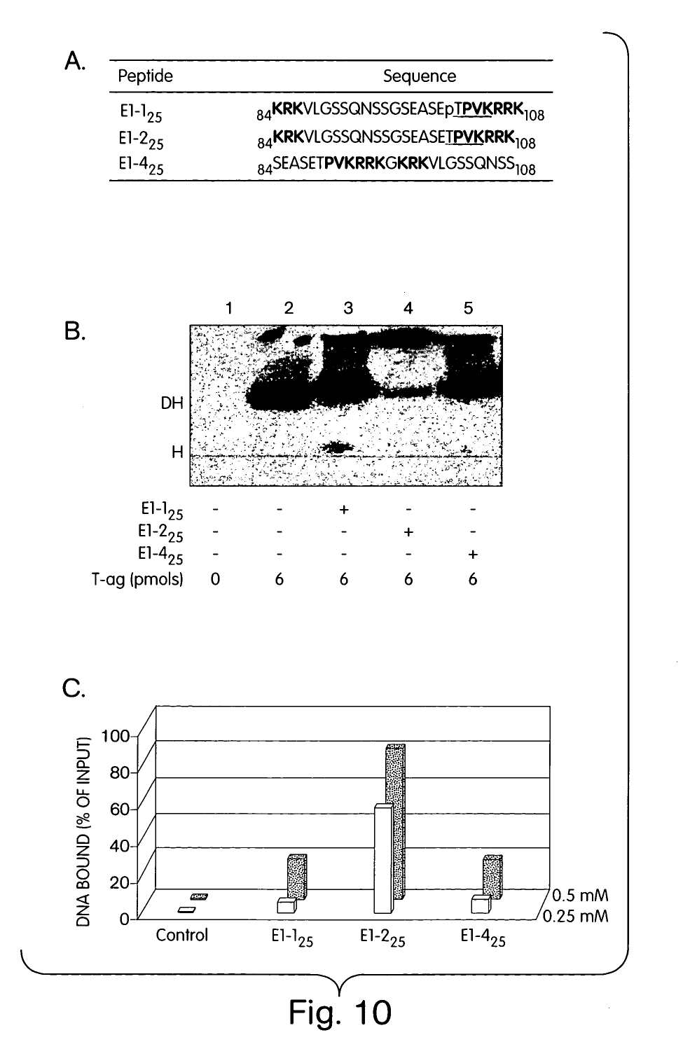

T-ag derived set of peptides centered on Thr124 are shown in FIG. 9A, the 13 mer (SEQ ID NOs: 12, 3, 13 and 14) and 17 mer sets (SEQ ID NOs: 15, 11, 16 and 17). In those sets, peptides designated as T-1 (SEQ ID NO: 12) and T-2 (SEQ ID NO: 3) were synthesized such that Thr124 is phosphorylated (for the peptide T-1), or is non-phosphorylated (for peptide T-2). Peptides T-3 (SEQ ID NO: 13) and T-4 (SEQ ID NO: 14) are controls designed by swapping the amino acids positioned on either side of Thr124 on peptides T-1 and T-2. Residues derived from the SV40 NLS are shown in bold; those forming the consensus Cyclin-Cdk recognition site (Moreno, S. et al., 1990, Cell 61:549-551) are underlined. In view of the presence of these features, these molecules are collectively referred to as the “Cdk/NLS” peptides. The 9 mer set of peptides set is also shown in FIG. 9A.

The 13-mer set of peptides was tested to determine if any of these peptides might interfere with T-ag assembly on a 64-bp oligonucleotide containing the SV40 core origin (FIG. 9 Panel B). In the absence of peptide, T-ag oligomerizes and forms hexamers and double hexamers on this DNA substrate (lane 2). Peptides T-1 ₁₃ (SEQ ID NO: 12), T-3₁₃ (SEQ ID NO: 13) and T-4₁₃ (SEQ ID NO: 14) have little or no effect on T-ag oligomerization (20 nmoles of each; final concentration of 1 mM; lanes 3, 5 and 6). However, in the presence of non-phosphorylated peptide T-2₁₃ (SEQ ID NO: 3), formation of the standard hexamer and double hexamer protein was inhibited.

Similar experiments were conducted with the “17 mer” sets of peptides. As with the 13-mer set of peptides, there was no inhibition of T-ag assembly by peptide T-1₁₇ (non-phosphorylated; SEQ ID NO: 15) or peptide T-3₁₇ (SEQ ID NO: 16; peptide concentration of 1 mM). However, T-ag assembly was completely blocked by peptide T-2₁₇ (SEQ ID NO: 11) and to a lesser extent by peptide T-4₁₇ (SEQ ID NO: 17). At a peptide concentration of 0.5 mM, peptide T-2₁₇ (SEQ ID NO: 11) inhibited T-ag oligomerization and peptide T-4₁₇ (SEQ ID NO: 17) did not. The members of the 9-mer set of peptides did not inhibit T-ag assembly, at any concentration tested.

These results show that inhibition of T-ag assembly into oligomers by a peptide requires that Thr124 be non-phosphorylated, and that the peptide have a length of at least 10 amino acid residues. Phosphorylated peptides can be of use as agents to reverse regulation of gene expression and DNA replication by non-phosphorylated peptides. It is here envisioned that therapeutic administration of a DNA binding peptide to inhibit DNA replication or transcription is reversible, and can be reversed by administration of a phosphorylated peptide having the same amino acid sequence.

Further, as shown in an example below, a peptide having a mutation in the SV40 T-ag sequence T124D (SEQ ID NO: 73) can also find application for reversing the effects of wild type non-phosphorylated inhibition of gene expression and DNA replication. The type of mutation of a peptide exemplified by T124D (SEQ ID NO: 73) having a negatively charged residue, which when substituted into a parental DNA-binding and inhibitory peptide that confers loss of DNA binding, is not limited to substitution of an aspartic acid, nor limited to position a position like T124 that can be phosphorylated, but can also be for example, a glutamic acid substitution, and can be located at another position of the inhibitory peptide. This type of mutation in a pharmaceutical peptide agent offers the considerable advantage in that it cannot be dephosphorylated by a cellular phosphatase in vivo, so that as a regulatory agent its effect can be sustained for the biological lifetime of the peptide or peptide derivative.

### Example 7

### DNA Binding by Peptides Derived from the Cdk/NLS Region of T-ag

One explanation for the ability of the unphosphorylated peptides to block T-ag oligomerization is that they are able to bind to DNA and block subsequent protein binding events. To test this hypothesis, a series of filter binding assays were conducted with the peptides derived from the “Cdk/NLS” region of T-ag. Results from these studies are presented in FIG. 9 Panel C.

Experiments with the 13-mer set of peptides demonstrated that peptide T-2₁₃ is able to bind to DNA. Other peptides here, such as peptide T-1₁₃ (SEQ ID NO: 12), did not bind to the oligonucleotide at significant levels. However, low levels of binding were observed with peptide T-4₁₃ (SEQ ID NO: 14) at high concentrations of peptide (e.g., 2 mM). DNA binding was also detected with the 17-mer set of peptides. At 0.5 mM, significant binding was observed only with peptide T-2₁₇ (SEQ ID NO: 11). Over the range of peptide concentrations tested (0.5 mM to 2 mM), the 9-mer peptides bound at background levels to the SV40 core origin containing oligonucleotide. Since peptide T-2₁₃ (SEQ ID NO: 3) bound relatively poorly at 0.5 mM, while peptide T-2₁₇ (SEQ ID NO: 11) bound significant levels of DNA at the same concentration, then peptide T-2₁₇ has higher affinity for this DNA than does peptide T-2₁₃ (SEQ ID NO: 3). The absence of significant increase in binding as a function of concentration of peptide T-2₁₇ (SEQ ID NO: 11) above 0.5 mM, indicates that the reactions conducted with this peptide at 1 mM and 2 mM were saturated. Consistent with this conclusion, additional members of the. 17-mer set of peptides (e.g., peptide T-1₁₇ (SEQ ID NO: 15) and peptide T-4₁₇ (SEQ ID NO: 17)) were observed to bind to DNA at higher peptide concentrations.

To further characterize the peptide-2/DNA interactions, a series of peptide band shift experiments were conducted (FIG. 9 Panel D). The initial set of reactions was performed in the presence of the 64-bp oligonucleotide containing the SV40 core origin (SEQ ID NO: 24, FIG. 9 Panel D, photograph 1); the position of the DNA substrate, in the absence of peptide, is indicated (lane 1). The reaction products formed when the 13-mer set of peptides were added to the reactions are indicated in lanes 2-5 (20 nmoles; 1 mM final concentration). These results show that only unphosphorylated peptide T-2₁₃ (SEQ ID NO: 3), among 13-mer peptides tested here, binds to DNA (lane 3).

To determine whether peptide binding depends upon a high degree of sequence specificity of the target DNA, the experiment in FIG. 9D photograph 1 was performed using a control oligonucleotide (SEQ ID NO: 29; the 64-bp enhancer control as described by Joo, W. S. et al., 1998, Mol. Cell. Biol. 18:2677-2687; FIG. 9 Panel D, photograph 2). Peptide T-2₁₃ (SEQ ID NO: 3; lanes 2-5) is able to bind to the 64-bp enhancer control. Therefore, binding of DNA by peptide T-2₁₃ is not highly sequence specific, an observation confirmed by additional filter binding assays. Moreover, as with reactions conducted in the presence of the core origin, other members of the 13-mer set of peptides did not bind to the 64-bp enhancer control (FIG. 9D photograph 2).

The reaction with peptide T-2₁₃ (SEQ ID NO: 3; lane 3) produced a larger amount of material retained in the sample application well than did any other peptides. This retention of material in the sample well shows that in addition to the standard peptide/DNA complex, larger aggregates were formed upon binding of this peptide to DNA.

The experiments presented in FIG. 9 Panel D were performed with the 17-mer set of peptides, and results similar to those with the 13-mer set were obtained. However, even at a final concentration of 2 mM, binding to DNA was not detected with the 9-mer set of peptides. The data in FIG. 9 demonstrate that peptides derived from the NLS region of T-ag found here to bind to DNA have the following characteristics: the peptides lack a phosphate at residue Thr124; the amino acid sequence is at least 13 amino acids long; and the sequence of amino acids has not been rearranged.

### Example 8

### A Peptide Containing the NLS from Bovine Papillomavirus E1 Binds to DNA

It was of interest to establish whether non-T-ag based Cdk/NLS peptides can also bind to DNA. The Bovine Papillomavirus (BPV) E1 NLS, extending between residues 84-108 of the E1 protein, has been characterized (Lentz, M. R. et al., 1993, J. Virol. 67:1414-1423). It is a representative member of the bipartite NLS elements (Jans, D. A. et al., 1996, Physiol. Rev. 76:651-685), and Thr102 of this protein is a putative site for phosphorylation by a cyclin/Cdk kinase (Lentz, M. R. et al., 1993, J. Virol. 67: 1414-1423).

Two 25 residue long peptides containing this region were designed and synthesized. Peptide E1-P1₂₅ contains a phosphate at Thr102 (SEQ ID NO: 18) and peptide E1-P2₂₅ (SEQ ID NO: 2) is unphosphorylated (FIG. 10 Panel A). As a control for peptide E1-P2₂₅, peptide E1-P4₂₅ (SEQ ID NO: 19) was designed by swapping the residues on either side of Gly 96 (FIG. 10 Panel A).

The data herein show that, as with the T-ag based peptides, the unphosphorylated E1 based peptide blocked T-ag assembly (FIG. 10 Panel B, lane 4), and the phosphorylated peptide does not (FIG. 10 Panel B, lane 3). Control peptide E1-P4₂₅ (SEQ ID NO: 19) was found not to block T-ag assembly (lane 5).

Further, a series of filter binding experiments were conducted to determine whether peptide E1-P2₂₅ (SEQ ID NO: 2), in addition to blocking T-ag assembly, also bound to DNA (FIG. 10 panel C). Of the three E1 based peptides that were synthesized, only unphosphorylated peptide E1-P2₂₅ (SEQ ID NO: 2) was found to bind to DNA. Surprisingly, this peptide binds at a relatively low concentration of peptide (final concentration of 0.25 mM); thus, it is a better DNA binder than the T-ag derived peptide T-2₁₇ (SEQ ID NO: 11).

That peptide E1-2₂₅ (SEQ ID NO: 2) is a relatively strong DNA binder was further demonstrated by performing successful EMSA experiments with peptide E1-2₂₅ in the absence of cross-linking with glutaraldehyde. In contrast, peptide E1-1₂₅ (SEQ ID NO: 18) which is phosphorylated on Thr 102, binds to DNA at levels similar to the control peptide E1-4₂₅ (SEQ ID NO: 19).

Thus, peptides derived from the NLS regions of each of the E1 and T-ag proteins bind to DNA in a manner that is regulated by phosphorylation, and is dependent on the sequence of the amino acid residues adjacent to the NLS.

### Example 9

### The Amino Acid Residue Dependence of Peptide-DNA Binding: Requirement for NLS but not Cyclin/Cdk Sequence

Examples herein show that phosphorylation of a Thr residue site regulates DNA binding of the peptides. Further data were obtained to address whether Thr124 (T124) is essential for DNA binding, and whether flanking residues, such as Pro125 or Pro126 (P125 or P126), and whether the integrity of the NLS sequence (at NLS residue Lys128, K128) are also required for DNA binding. Phosphorylation of Thr-Pro motifs alters the cis/trans isomerization rate of a prolyl bond (Zhou, X. Z. et al., 1999, Cell. Mol. Life Sci. 56:788-806). Moreover, the presence of a second proline is known to favor the cis conformation (Fischer, G., 2000, Chem. Soc. Rev. 29:119-5 127).

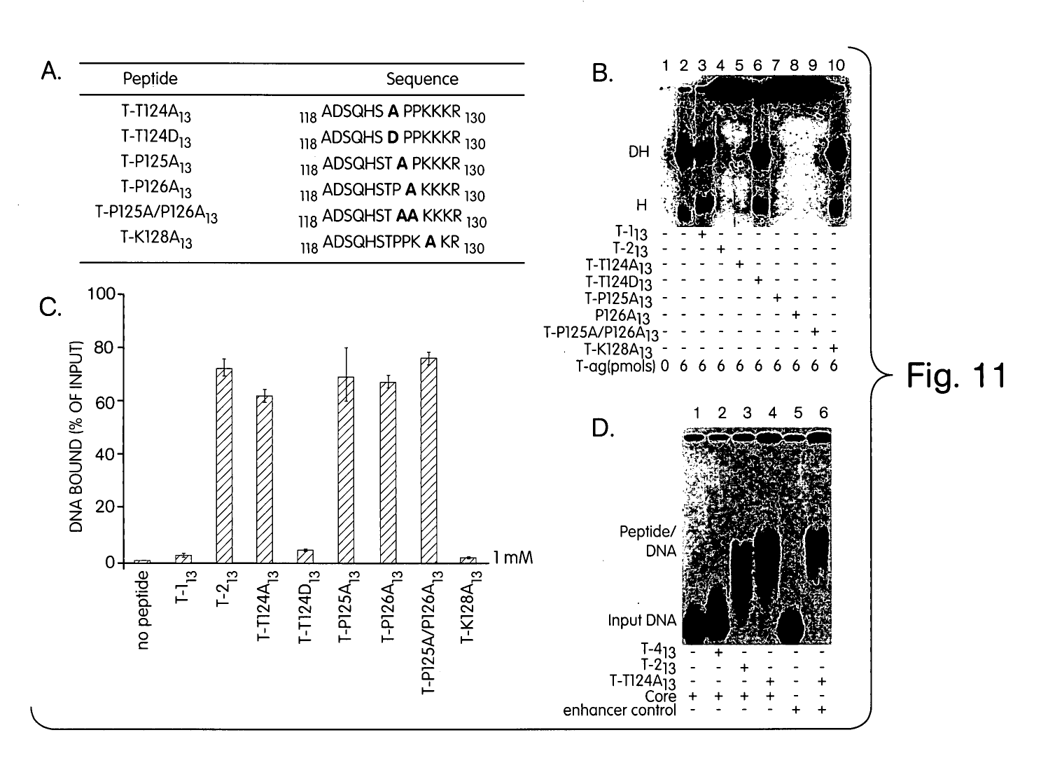

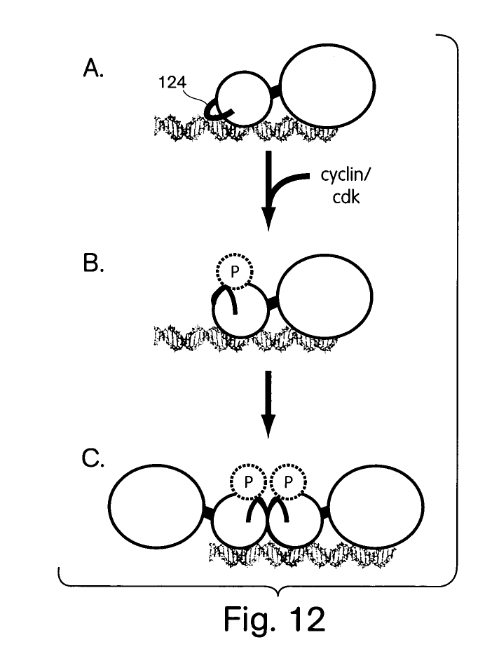

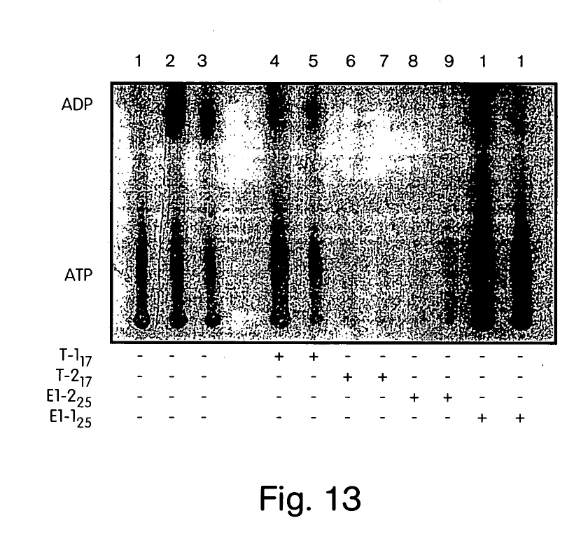

Derivatives of peptide T-2₁₃ (SEQ ID NO: 3) containing alanine substitutions at the indicated locations were designed and synthesized (FIG. 11 Panel A), to test whether these residues are essential for DNA binding using the 64 base pair SV40 core oligonucleotide (SEQ ID No: 24). The mutant peptides are termed T124A₁₃, P125A₁₃, P126A₁₃, and P125A/P126A₁₃ (SEQ ID NOs: 20-23, respectively) and T124D (SEQ ID NO:73) and K128A (SEQ ID NO: 74), in sequences shown in FIG. 11 Panel A. The single mutant and double mutant P125A/P126A₁₃ peptides were synthesized to determine the effects of each of these mutations on ability of the resulting peptide to bind DNA.

Reactions were designed to determine also whether the mutant set of peptides had an effect on T-ag oligomerization, and were analyzed by gel electrophoresis as shown in FIG. 11B. Controls lacking peptides were conducted in the absence of T-ag protein (lane 1), and in the presence of T-ag (lane 2, 6 pmols). Test reactions were conducted in the presence of T-ag with phosphorylated peptide T-1₁₃ (SEQ ID NO: 12; lane 3), or with non-phosphorylated peptide T-2₁₃ (SEQ ID NO: 3; lane 4).

Results show that non-phosphorylated peptide T-2₁₃ inhibited T-ag oligomerization, while phosphorylated peptide T-1₁₃ did not. Without being limited by any particular mechanism, the increased size of the double hexamers observed in the presence of phosphorylated peptide T-1₁₃ (lane 3 compared to lane 2) shows that the peptide in fact binds to T-ag but does not disrupt oligomerization.

The reaction products formed in the presence of T-ag (6 pmols) and the mutant peptides are shown in lanes 5-10. As with peptide T-2₁₃ (SEQ ID NO: 3), each of the mutant peptides having an A in place of T124, P124, P125, or both P125 and P126, disrupted T-ag oligomerization.

However, a mutant peptide having a substitution of negatively charged aspartic acid (D) at position 124 (mutant T124D, lane 6; SEQ ID NO: 73) failed to disrupt oligomerization. As this mutation changes the peptide not merely by removing the T, but also by adding a negative charge, this substitution makes the peptide behave like a phosphorylated peptide. These results show, surprisingly, that a mutant peptide having a negative charge such as mutant T124D can substitute for a phosphorylated peptide as an agent to provide release of inhibition by a non-phosphorylated peptide bound to DNA.

Loss of one of the basic amino acids in the NLS also disrupts inhibition of oligomerization, as shown in lane 10 with the mutant peptide K128A (SEQ ID NO: 74). These data show that binding to DNA of the peptides herein, demonstrated by inhibition of oligomerization, is conferred in wild type sequences by the NLS, and that the presence of a Cyclin/Cdk site is not essential for this inhibition.

Filter binding assays were performed to determine the amount of each of the mutant set of peptides that binds to DNA. The data shown in FIG. 11 Panel C, obtained from assays conducted with 20 nmoles of peptide (1 mM final concentration), indicate that with the exception of T124D and K128A, the mutant peptides T124A₁₃, P125A₁₃, P126A₁₃ and double mutant P125A/P126A₁₃ (SEQ ID NOs: 20, 21, 22 and 23, respectively) bind to DNA as well as does wild type peptide T-2₁₃ (SEQ ID NO: 3). These data show that the presence of any of the amino acid residues T124, P125 or P126 are not necessary for binding. These data further show that a basic residue at position 128 is required for binding, and that substitution of an amino acid having a negative charge at position 124 disrupts DNA binding.

To determine directly whether the mutant peptides form peptide/DNA complexes similarly to those formed with peptide T-2₁₃, peptides were tested in a “peptide-gel shift” assay (FIG. 11 Panel D). The reactions in lanes 1-4 were conducted with the 64-bp SV40 core origin oligonucleotide (SEQ ID NO: 24). Results obtained with wild type peptide are shown in lane 3, and results with mutant T124A₁₃ are shown in lane 4 of FIG. 11 Panel D. These data show that the T124A mutation does not affect DNA binding of peptides, as indicated by observation of comparable mobility shifts in both lanes. The control reaction in lane 1 was performed in the absence of peptide, while the reactions displayed in lanes 2 and 3 were conducted in the presence of peptide T-4₁₃ (SEQ ID NO: 14) and peptide T-2₁₃ (SEQ ID NO: 3), respectively, which served as negative and positive controls, respectively. The reaction in lane 4 was conducted with peptide T124A₁₃.

The results show that mutant peptide T124A₁₃ (SEQ ID NO: 20) bound to DNA and formed a complex, similar to results obtained with wild type peptide T-2₁₃ (SEQ ID NO: 3).The reactions in landes 5 and 6 were conducted with the 64 bp enhancer control oligonucleotide (SEQ ID NO: 29). The reaction in lane 6 demonstrates that, as with peptide T-2₁₃ (SEQ ID NO: 3), binding of peptide T124A₁₃ (SEQ ID NO: 20) to DNA requires little demonstrable nucleotide sequence specificity. It is likely that a combination of charge and sequence underlies binding of peptides to DNA.

These data demonstrate that the T-ag amino acid residues at positions Thr124, Pro125 and Pro126 are not essential for DNA binding, an observation consistent with these three residues having primarily a regulatory role. However, the strength of the NLS is an important determinant in DNA binding and other inhibitory processes described herein. FIG. 12 shows a model of the mechanism of binding and oligeromerization as related to phosphorylation at a Thr residue.

### Example 10

Peptides Bind to T-ag and Inhibit Helicase ATPase Activity in a Phosphorylation-Dependent Manner

T-ag possesses a helicase activity that requires ATP hydrolysis for its activity. A method for measuring ATP hydrolysis involves incubating T-ag with a ³²P-labeled ATP, cross-linking with gluteraldehyde, removing unincorporated nucleotides by passing the reaction products over a Bio-Spin P-30 column, drying the eluate in a Speedvac, resuspending the samples in formic acid, and performing thin layer chromatography on PEI (polyethyleneimine) plates. Using autoradiography, the amount of ATP, ADP and AMP associated with T-ag was determined.

Since peptides herein affect oligomerigation of T-Ag, the effect of these peptides on the T-ag ATPase assay was tested (FIG. 13). The reactions in lanes 2 and 3 are positive controls in the absence of added peptides, to show that T-ag bound to ATP produced ADP. Lanes 2 and 3 demonstrate that the helicase ATPase of T-Ag produces ADP (the product of ATP hydrolysis).

The reaction products observed in the presence of both T-ag and phosphorylated peptide T-1₁₃ (SEQ ID NO: 15) are shown in lanes 4 and 5. Little difference was observed between the products formed in these reactions, and those formed in the T-ag control (lanes 2 and 3). It is concluded that the phosphorylated peptide did not modulate the ATPase of T-ag activity.

In contrast, effect on the reaction of peptide T-2₁₃ (SEQ ID NO: 3; unphosphorylated), shows that this peptide inhibited the ATPase of T-ag (FIG. 13, lanes 6 and 7). The data in lanes 8-11 demonstrate that the same effect is observed with the E1 peptides. The unphosphorylated peptide E1-2₂₅ (SEQ ID NO: 2) blocked the degradation of ATP by T-ag (FIG. 13, lanes 8 and 9), while the phosphorylated peptide E1-1₂₅ (SEQ ID NO: 18) had no effect. These data show that unphosphorylated peptides inhibit helicase ATPase activity by T-ag, and therefore must interact with a region of this protein in a manner that interferes with the helicase enzyme active site.

Results herein demonstrate that peptides derived from the Cdk/NLS regions of SV40 T-ag and BPV E1, when unphosphorylated, have ability both to bind to DNA and also to bind to T-ag. Thus NLS regions confer at least two important and distinct properties, nuclear entry and cell cycle regulated DNA binding.

Advantages of Cdk/NLS peptides with respect to other DNA-binding proteins such as Zn finger and a-helix proteins, are that the peptides herein are smaller than those proteins or other DNA binders heretofore described, and that the peptides herein are the only members of this group for which binding to DNA is regulated by phosphorylation.

Phosphorylation of T-ag derived peptides on Thr124, E1 derived peptides on Thr102, greatly reduces binding of peptides to DNA and to T-ag. Therefore, phosphorylation confers a regulatory switch, that down regulates DNA binding and T-ag binding by the peptides.

Without being bound by any particular mechanism, it is possible that upon phosphorylation, the Cdk/NLS peptides no longer bind to DNA owing to electrostatic repulsion. Alternatively, phosphorylation induces structural changes that disrupt DNA binding. This model is supported by studies indicating that phosphorylation of Ser/Thr residues regulates rate of cis/trans isomerization of Ser/Thr-Pro bonds (Fischer, G., 2000, Chem. Soc. Rev. 29:119-127; and Zhou, X. Z. et al., 1999, Cell. Mol. Life Sci. 56:788-806).

### Example 11

### Role of Cis-Trans Prolyl Isomerization in Regulation of Double Hexamer Formation

The phosphorylated and unphosphorylated forms of the Cdk/NLS peptides can have different configurations, and thus different affinities for DNA. Phosphorylation of the Ser/Thr-Pro motif creates a substrate for the peptidyl-prolyl isomerase Pin1 (Yaffe, M. B. et al., 1997, Science 278: 1957-1960). Cis/trans isomerization rate in vivo of the Cdk/NLS motifs can be regulated by peptidyl-prolyl isomerases such as Pin1. Phosphorylation of Thr124 is not required for assembly of the initial hexamer. In contrast, on DNA origin sub-fragments containing single hexamer assembly units, phosphorylation of Thr124 is required to promote the assembly of the second hexamer (Barbaro, B. A. et al., 2000, J. Virol. 74:8601-8613).

The region of T-ag encompassing the Cdk/NLS motif, when unphosphorylated, can bind to DNA (FIG. 12). When bound to DNA, the Cdk/NLS motif blocks the assembly of the second hexamer to the second GAGGC pentanucleotide. However upon phosphorylation of Thr124, residues comprising the Cdk/NLS motif no longer bind DNA and a second hexamer can assemble, completing oligeromerization of this protein from a single hexamer to the double hexamer form. The regulation of papillomavirus DNA replication is similar. A role for peptidyl-prolyl isomerases, such as Pin I in these regulatory events is indicated.

### Example 12

### Synthesis and Testing of Peptidomimetic Compounds

The peptides described herein are envisioned as the basis of design of peptidomimetic compounds, which can be synthesized and tested for retention of level of activity possessed by the peptide on which the peptidomimetic is based. A peptidomimetic compound can be of low molecular weight (e.g., 300-600, 400-900, or 300-2000 daltons mol weight), and can resemble a short amino acid sequence of any of the peptides herein in shape and charge. The peptidomimetic can bind to a site on the biological target molecule, and can have affinity and specificity (FIG. 12) to that of the peptide on which it is based.

A peptidomimetic of the present invention can bind to DNA and inhibit DNA replication. Similarly, a peptidomimetic can bind to and inhibit the ATPase activity of a helicase. Criteria for the design and synthesis of peptidomimetic compounds, including suitable components and reactions, are described in U.S. Patent Nos. 6,080,838, issued Jun. 27, 2000; 6,245,742, issued Jun. 12, 2001; 6,291,640, issued Sep. 18, 2001; and 5,846,944, issued Dec. 8, 1998. Peptidomimetic compounds are further tested for additional pharmacological activities, such as absorption following oral administration to animals, distribution into various tissue types, rate of metabolism and activity of metabolized products, excretion route and kinetics, and toxicology.

### Example 13

### Design of Polyvalent Peptides and Peptidomimetic Compounds

Affinity of any of the aforementioned peptides and peptide-based inhibitors for DNA, and ability to inhibit DNA replication, can be increased by preparing a longer peptide having an increased number of amino acid residues, for example, with two copies of the amino acid sequence of the peptide arranged in tandem in the longer “dimeric” peptide (see Mourez et al. 2001 Nature Biotech 19:958-961). Design of a peptide having additional copies of the sequence are envisioned, for example, a trimeric or tetrameric sequence compared to the initial peptide. Further, an increased length of the peptide need not be limited to integral increments, as a portion of the amino acid sequence of the initial peptide can also be added to that of the initial peptide.

### Example 14

### Analyzing Cdk/NLS Libraries Binding to Target DNA by Mass-Spectroscopy

Libraries of peptides can be made having two variable positions in an amino acid sequence. With 20 possible amino acids at each position, 400 variation members of a library are obtained. Similarly, three variable positions generates 8,000 members. The minimal level of detection is about one picomole of peptide in about 5 microliters (concentration of each compound about 0.2 micromolar) which can be obtained for libraries of this complexity.

Peptide libraries based on the Cdk/NLS motifs are prepared at the Tufts University Core Facility using standard methods. In two separate reactions, samples of the peptide libraries are mixed with either a target DNA (e.g. an origin of replication from a polyoma virus) or a duplex DNA fragment of random sequence having the same number of nucleotide base pairs. These target DNA and control sequences are synthesized with a bindable handle (e.g. a biotin moiety) to enable isolation of DNA/peptide complexes. Once isolated, peptide/DNA complexes are removed from the DNA and analyzed by mass-spectroscopy (LCMS using electrospray ionization and a Finnigan LCQ ion trap spectrometer).

Peptide sequences are deduced from the observed mass of the ions or, when required, by analysis of the collision induced fragments. This analysis establishes the structure of the particular peptides that bind to DNA targets, i.e., their amino acid sequence, and the affinity of the peptides to the target DNA, thereby determining whether particular Cdk/NLS peptides preferentially bind to the DNA target. By this method, specific binding of peptides to DNA targets can be determined, and the chemical identity of those peptides having the greatest affinity and specificity can be established.

Specific binding of peptides and their peptidomimetics to target DNA sequences will enable such peptides and peptidomimetics to act to down-modulate expression of one or more of specific genes (“target” gene or genes) that carry the target DNA sequence. This use is envisioned to reduce or eliminate gene expression of the target gene, similar to use of interfering RNA molecules (RNAi), for analysis of phenotypes resulting from such reduction or elimination, and for potential therapeutic purposes, for example, in diseases such as Parkinson's disease.

TABLE 2
A.

| | 9-mer | 13-mer | 17-mer |
|---|---|---|---|
| Peptide 1 | 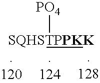 | 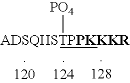 | 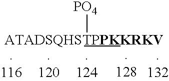 |
| Peptide 2 | 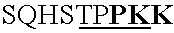 | 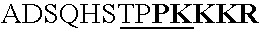 | 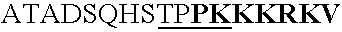 |
| Peptide 3 | 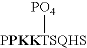 | 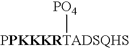 | 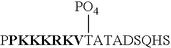 |
| Peptide 4 | PPKKTSQHS | PPKKKRTADSQHS | PPKKKRKVTATADSQHS |

B.

| | |
|---|---|
| BP-E1 | 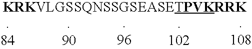 |
| BP-E1 controll | SEASETPVKRRKGKRKVLGSSQNSS |

Table 2. Peptides derived from the NLS regions of SV40 T-ag and Bovine Papillomavirus E1. A). Amino acid sequences of three sets of peptides, centered on Thr124, derived from SV40 T-ag. The sizes of the peptides are indicated (i.e., 9-mer, 13-mer and 17-mer); the peptide numbering system is based on position of the corresponding residues in full length T-ag. In a given set, peptides 1 and 2 are identical except that peptide I is phoshorylated on Thr124 and peptide 2 is not. Control peptides 3 and 4 were derived from peptides 1 and 2 by inverting the residues found on each side of Thr124. Residues that comprise the T-ag NLS are shown in bold. Amino acid residues underlined in the wild type peptides identify the recognition motif for the cyclin/cyclin dependent kinase. The 9-mer peptides listed from the top are SEQ ID NO: 25, SEQ ID NO: 26, SEQ ID NO: 27, and SEQ ID NO: 28. The 13 mer peptides listed from the top are SEQ ID NO: 12, SEQ ID NO: 3, SEQ ID NO: 13, and SEQ ID NO: 14. The 17-mer peptides listed from the top are SEQ ID NO: 15, SEQ ID NO: 11, SEQ ID NO: 16, and SEQ ID NO: 17. B). Peptides derived from Bovine papillomavirus E1 in the NLS region. The bipartite NLS found in Bovine papillomavirus E1 is present in a 25 residue long peptide termed BP-E1₂₅ (SEQ ID NO: 2). The peptide numbering system is based on positions of the corresponding residues in Bovine papillomavirus E1. A control peptide termed BP-E1 control ₂₅ (SEQ ID NO: 19) was formed by inverting the residues on each side of the glycine residue at position 96. Residues in bold identify the Bovine Papillomavirus E1 NLS and those that are underlined identify the putative recognition motif for the cyclin/cyclin Cdk complex (Lentz, M. R., et al., 1993, J.Virol. 67:1414-1423).

What is claimed is:

1. An isolated peptide comprising an amino acid sequence of a nuclear localization signal (NLS), wherein the peptide has a length of at least about 8 amino acids to about 50 amino acids, and binds directly to a DNA molecule.

2. The peptide of claim 1, further comprising a cyclin/Cdk recognition site.

3. The peptide of either of claim 1 or claim 2, wherein the peptide is not phosphorylated.

4. The peptide of claim 1, wherein the peptide does not comprise a phosphorylation site.

5. The peptide of claim 1, wherein the peptide does not contain a residue that is a threonine, a serine, or a tyrosine residue.

6. The peptide of claim 5, wherein the cyclin/Cdk recognition site comprises a threonine (T) residue.

7. The peptide of claim 6, wherein the cyclin/Cdk recognition site is not phosphorylated.

8. The peptide of claim 1, wherein the peptide inhibits replication of the DNA molecule.

9. The peptide of claim 8, wherein the DNA molecule comprises an origin of replication.

10. The peptide of claim 1, wherein the peptide inhibits transcription of the DNA molecule.

11. The peptide of claim 1, wherein the DNA molecule comprises a viral DNA sequence.

12. The peptide of claim 1, wherein the DNA molecule comprises a nucleotide sequence from a eukaryotic cell.

13. The peptide of claim 11, wherein the DNA molecule comprises a nucleotide sequence from a papovavirus.

14. The peptide of claim 11, wherein the amino acid sequence of the DNA molecule comprises a nucleotide sequence from a papillomavirus.

15. The peptide of claim 1, wherein the amino acid sequence of the peptide comprises a papillomavirus E1 gene NLS.

16. The peptide of claim 14, wherein the DNA molecule comprises a nucleotide sequence from a papillomavirus strain 16 or 18.

17. The peptide of claim 11, wherein the DNA molecule comprises a nucleotide sequence from a BK virus or a JC virus.

18. The peptide of claim 1 produced by chemical synthesis.

19. The peptide of claim 18 comprising at least one non-peptidic bond.

20. The peptide of claim 1 in a pharmaceutically acceptable carrier.

21. A method of inhibiting DNA replication of a DNA virus, comprising contacting a nucleic acid sequence of a viral DNA molecule with the peptide of claim 1.

22. An isolated peptide comprising an amino acid sequence selected from the group consisting of:

| | |
|---|---|
| TKRALPNNTSSSPQPKKKPL, | (SEQ ID NO: 1) |
| KRKVLGSSQNSSGSEASETPVKRRK, | (SEQ ID NO: 2) |
| KKRRKRVKLVGPSTSEQSNASESSG, | (SEQ ID NO: 30) |
| ADSQHSTPPKKKR, | (SEQ ID NO: 3) |
| KLWLHGTPKKNCI, | (SEQ ID NO: 4) |
| KSFLKGTPKKNCL, | (SEQ ID NO: 5) |
| GSQHSTPPKKKR, | (SEQ ID NO: 6) |
| QSSYTCTPPKRKK, | (SEQ ID NO: 7) |
| QQSHHNTTPKKPP, | (SEQ ID NO: 8) |
| QSSFNATPPKKAR, | (SEQ ID NO: 9) |
| PARSQATPPKKKA, | (SEQ ID NO: 10) |
| ATADSQHSTPPKKKRKV, | (SEQ ID NO: 11) |
| SQHSTPPKK; | (SEQ ID NO: 26) |
| PPKKTSQHS; | (SEQ ID NO: 28) |
| ADSQHSDPPKKKR; | (SEQ ID NO: 73) |
| and | |
| ADSQHSTPPKAKR. | (SEQ ID NO: 74) |

23. An isolated peptide comprising amino acid sequence KRKVLGSSQNSSGSEASETPVKRRK (SEQ ID NO: 2).

24. An isolated peptide comprising amino acid sequence ADSQHSTPPKKKR (SEQ ID NO: 3).

25. An isolated peptide comprising amino acid sequence ATADSQHSTPPKKKRKV (SEQ ID NO: 11).

26. An isolated peptide comprising amino acid sequence KKPRKRVKLVGPSTSEQSNASESSG (SEQ ID NO: 30).

27. An isolated peptide comprising a polyvalent multimer of the amino acid sequences of the SEQ ID NOs according to claim 22.

28. An isolated peptide having an amino acid sequence of a nuclear localization signal (NLS) and a site for cyclin/Cdk phosphorylation, the peptide capable of binding to a DNA molecule and inhibiting replication or transcription of the DNA molecule.

29. The peptide of claim 28, the peptide being further capable of inhibiting assembly of a multimeric DNA-binding protein.

30. The peptide of claim 28, further comprising a negatively charged amino acid.

31. An isolated peptide comprising an amino acid sequence selected from the group consisting of:

| | |
|---|---|
| ADSQHSpTPPKKKR, | (SEQ ID NO: 12) |
| PPKKKRpTADSQHS, | (SEQ ID NO: 13) |
| PPKKKRTADSQHS, | (SEQ ID NO: 14) |
| ATADSQHSpTPPKKKRKV, | (SEQ ID NO: 15) |
| PPKKKRKVpTATADSQHS, | (SEQ ID NO: 16) |
| PPKKKRKVTATADSQHS, | (SEQ ID NO: 17) |
| KRKVLGSSQNSSGSEASEpTPVKRRK, | (SEQ ID NO: 18) |
| SEASETPVKRRKGKRKVLGSSQNSS, | (SEQ ID NO: 19) |
| ADSQHSAPPKKKR, | (SEQ ID NO: 20) |
| ADSQHSTAPKKKR, | (SEQ ID NO: 21) |
| ADSQHSTPAKKKR, | (SEQ ID NO: 22) |
| ADSQHSTAAKKKR, | (SEQ ID NO: 23) |
| SQHSpTPPKK; | (SEQ ID NO: 25) |
| and | |
| PPKKpTSQHS, | (SEQ ID NO: 27) |

wherein pT indicates a phosphorylated threonine residue.

32. An isolated peptide having an amino acid sequence of a nuclear localization signal (NLS), wherein the peptide binds to a helicase.

33. The peptide of claim 32, wherein the peptide inhibits ATPase activity of the helicase.

34. The peptide of claim 32, wherein the helicase is a viral protein.

35. The peptide of claim 32, wherein the helicase is involved in initiation of viral DNA replication.

36. A method of identifying a derivative of a parent nucleotide sequence, the nucleotide sequence encoding a parent peptide comprising an NLS sequence of amino acids, the derivative encoding a variant peptide having increased affinity for a target DNA nucleotide sequence compared to the parent peptide, the method comprising:
displaying the parent peptide on a coat protein of a phage by expressing the parent nucleic acid sequence on the phage chromosome;
mutagenizing the parent nucleotide sequence encoding residues of the parent peptide that form a surface to bind to the target DNA sequence, to produce a resulting library of variant peptides displayed on the phage; and
adsorbing the library to and selectively eluting the library from a substrate comprising immobilized target DNA, such that peptides having increasingly greater affinity for the target DNA are eluted successively from the substrate, to obtain a phage clone displaying the variant peptide having increased affinity for the target DNA sequence compared to the parent peptide.

37. The method of claim 36, wherein the peptide comprises at least about 8 amino acid residues to about 50 amino acid residues.

38. The method of claim 36, wherein absorbing the library to the immobilized substrate is providing at least about 20 nM of peptide equivalents.

39. The method of claim 36, wherein the target DNA nucleotide sequence comprises an origin of replication of a virus.

40. The method of claim 39, wherein the virus is a eukaryotic cell pathogen.

41. The method of claim 36, further comprising determining the nucleotide sequence encoding the peptide.

42. The method of claim 41, further comprising synthesizing the peptide in vitro.

43. The method of claim 42, wherein the peptide has at least one chemical modification.

44. The method of claim 43, wherein the modification is a D-amino acid.

45. The method of claim 43, wherein the modification is a non-peptide bond.

46. The method of claim 43, wherein the modification is an amino acid substitution by a non-naturally occurring amino acid analogue.

47. The peptide of claim 43, wherein the peptide with the chemical modification has greater binding affinity for the target DNA than the parent peptide.

48. The library of mutagenized peptides of claim 36.

49. A variant peptide obtained by the method of claim 36.

50. A method of inhibiting assembly of a multimeric DNA binding protein to a mature form in a cell, the method comprising:
contacting the cell with a peptide having an amino acid sequence of a nuclear localization signal and ability to bind to the multimeric protein; and
providing conditions under which the peptide binds to the protein, such that assembly to the mature form is inhibited.

51. A method of screening a plurality of chemical compounds to identify an agent having ability to inhibit DNA replication, the method comprising:
providing a first reaction having a DNA molecule, at least one of the plurality of compounds, substrates for DNA synthesis, and a eukaryotic cell extract, under conditions such that in a second reaction which is a control lacking the compound but is otherwise identical to the first reaction, the DNA molecule is replicated; and
comparing the amount of DNA replication in the first reaction with that in the second reaction and in a third reaction having a Cdk/NLS peptide instead of the compound and is otherwise identical to the first reaction, such that an amount of replication in the first reaction that is less than the amount in the second reaction indicates that the chemical is an inhibitor of DNA replication, and the amount of replication in the first reaction compared to that in the third reaction indicates the relative extent of inhibitory activity of the chemical.

52. A method of screening a chemical compound to identify an agent having ability to inhibit a helicase ATPase, the method comprising:
providing a first reaction having the helicase, the compound, and a substrate for the ATPase activity, under conditions such that in a second reaction which is control lacking the compound but is otherwise identical to the first reaction, the helicase has ATPase activity; and
comparing the amount of ATPase activity in the first and second reactions with that in a third reaction having a Cdk/NLS peptide instead of the compound and is otherwise identical to the first reaction, such that an amount of ATPase activity in the first reaction that is less than in the second reaction indicates that the compound is an agent which is a ATPase activity inhibitor, and the amount of ATPase activity in the first reaction compared to the third reaction indicates the extent of inhibitory activity of the compound.
